SEQUENCE LISTING (56 sequences)

SEQ ID NO: 1 - protein, 11 aa, Artificial Sequence
  Synthetic Peptide
GSSSGRGDSPA

SEQ ID NO: 2 - protein, 7 aa, Artificial Sequence
  Synthetic peptide
CRGDDVC

SEQ ID NO: 3 - protein, 5 aa, Artificial Sequence
  Synthetic peptide
RGDFK

SEQ ID NO: 4 - protein, 7 aa, Artificial Sequence
  Synthetic peptide
GRGDFSK

SEQ ID NO: 5 - protein, 6 aa, Artificial Sequence
  Synthetic peptide
  misc_feature (3)..(4): Xaa can be any naturally occurring amino acid
RKXXRK

SEQ ID NO: 6 - protein, 6 aa, Artificial Sequence
  Synthetic peptide
RGDDFL

SEQ ID NO: 7 - protein, 7 aa, Artificial Sequence
  Synthetic peptide
GRGDFSL

SEQ ID NO: 8 - protein, 7 aa, Artificial Sequence
  Synthetic peptide
  misc_feature (5)..(6): Xaa can be any naturally occurring amino acid
RTDLXXL

SEQ ID NO: 9 - protein, 12 aa, Artificial Sequence
  Synthetic peptide
YHWYGYTPQNVI

SEQ ID NO: 10 - protein, 7 aa, Artificial Sequence
  Synthetic peptide
EHGAMEI

SEQ ID NO: 11 - protein, 8 aa, Artificial Sequence
  Synthetic peptide
DELARLLT

SEQ ID NO: 12 - protein, 7 aa, Artificial Sequence
  Synthetic peptide
YNPTTYQ

SEQ ID NO: 13 - protein, 6 aa, Artificial Sequence
  Synthetic peptide
SVDNPH

SEQ ID NO: 14 - protein, 6 aa, Artificial Sequence
  Synthetic peptide
KCCYSL

SEQ ID NO: 15 - protein, 7 aa, Artificial Sequence
  Synthetic peptide
LTVSPWY

SEQ ID NO: 16 - protein, 12 aa, Artificial Sequence
  Synthetic peptide
KYLAYPDSVHIW

SEQ ID NO: 17 - protein, 12 aa, Artificial Sequence
  Synthetic peptide
WQPDTAHHWATL

SEQ ID NO: 18 - protein, 8 aa, Artificial Sequence
  Synthetic peptide
DFSRYLWS

SEQ ID NO: 19 - protein, 11 aa, Artificial Sequence
  Synthetic peptide
VSNKYFSNIHW

SEQ ID NO: 20 - protein, 14 aa, Artificial Sequence
  Synthetic peptide
EQRLGNQWAVGHLM

SEQ ID NO: 21 - protein, 8 aa, Artificial Sequence
  Synthetic peptide
QWAVGHLM

SEQ ID NO: 22 - protein, 8 aa, Artificial Sequence
  Synthetic peptide
FCFWKTCT

SEQ ID NO: 23 - protein, 8 aa, Artificial Sequence
  Synthetic peptide
DWMGWMDF

SEQ ID NO: 24 - protein, 12 aa, Artificial Sequence
  Synthetic peptide
QLYENKRRPYIL

SEQ ID NO: 25 - protein, 6 aa, Artificial Sequence
  Synthetic peptide
RRPYIL

SEQ ID NO: 26 - protein, 7 aa, Artificial Sequence
  Synthetic peptide
HAIYPRH

SEQ ID NO: 27 - protein, 8 aa, Artificial Sequence
  Synthetic peptide
CPQPRPLC

SEQ ID NO: 28 - protein, 12 aa, Artificial Sequence
  Synthetic peptide
HTMYYHHYQHHL

SEQ ID NO: 29 - protein, 7 aa, Artificial Sequence
  Synthetic peptide
ATWLPPR

SEQ ID NO: 30 - protein, 16 aa, Artificial Sequence
  Synthetic peptide
NGYEIEWYSWVTHGMT

SEQ ID NO: 31 - protein, 17 aa, Artificial Sequence
  Synthetic peptide
RRLFYKKVGLFYKKVRR

SEQ ID NO: 32 - protein, 12 aa, Artificial Sequence
  Synthetic peptide
EWLSPNLAPSVR

SEQ ID NO: 33 - protein, 12 aa, Artificial Sequence
  Synthetic peptide
SNEWIQPRLPQH

SEQ ID NO: 34 - protein, 12 aa, Artificial Sequence
  Synthetic peptide
TNYLFSPNGPIA

SEQ ID NO: 35 - protein, 15 aa, Artificial Sequence
  Synthetic peptide
APYAPYCVYRGSWSC

SEQ ID NO: 36 - protein, 15 aa, Artificial Sequence
  Synthetic peptide
KYLKYLPYWPVLSSL

SEQ ID NO: 37 - protein, 15 aa, Artificial Sequence
  Synthetic peptide
VTMVTMEAINLAFPG

SEQ ID NO: 38 - protein, 13 aa, Artificial Sequence
  Synthetic peptide
TYYCCTYYWPLPC

SEQ ID NO: 39 - protein, 12 aa, Artificial Sequence
  Synthetic peptide
YSAYPDSVPMMS

SEQ ID NO: 40 - protein, 12 aa, Artificial Sequence
  Synthetic peptide
SWLAYPGAVSYR

SEQ ID NO: 41 - protein, 7 aa, Artificial Sequence
  Synthetic peptide
IPTLPSS

SEQ ID NO: 42 - protein, 7 aa, Artificial Sequence
  Synthetic peptide
YRAPWPP

SEQ ID NO: 43 - protein, 7 aa, Artificial Sequence
  Synthetic peptide
SSPSTSY

SEQ ID NO: 44 - protein, 7 aa, Artificial Sequence
  Synthetic peptide
AHKMGTP

SEQ ID NO: 45 - protein, 5 aa, Artificial Sequence
  Synthetic peptide
CRKEA

SEQ ID NO: 46 - protein, 12 aa, Artificial Sequence
  Synthetic peptide
GRKKRRQRRPPQ

SEQ ID NO: 47 - protein, 34 aa, Artificial Sequence
  Synthetic peptide
MCIPCFTTNPNMAAKCNACCGSRRGSCRGPQCIC

SEQ ID NO: 48 - protein, 35 aa, Artificial Sequence
  Synthetic peptide
CGPCFTTDANMARKCRECCGGIGKCFGPQCLCNRI

SEQ ID NO: 49 - protein, 32 aa, Artificial Sequence
  Synthetic peptide
CGPCFTTDHQMEQKCAECCGGIGKCYGPQCIC

SEQ ID NO: 50 - protein, 29 aa, Artificial Sequence
  Synthetic peptide
VSCEDCPDHCSTQKARAKCDNDKCVCEPI

SEQ ID NO: 51 - protein, 13 aa, Artificial Sequence
  Synthetic peptide
DEMEFTEAESNMN

SEQ ID NO: 52 - protein, 7 aa, Artificial Sequence
  Synthetic peptide
CFDWKTC

SEQ ID NO: 53 - protein, 7 aa, Artificial Sequence
  Synthetic peptide
CYDWKTC

SEQ ID NO: 54 - protein, 8 aa, Artificial Sequence
  Synthetic peptide
AAYDWLTF

SEQ ID NO: 55 - protein, 6 aa, Artificial Sequence
  Synthetic peptide
PFDYLI

SEQ ID NO: 56 - protein, 7 aa, Artificial Sequence
  Synthetic peptide
KLAKLAK

(19) United States
(12) Patent Application Publication
(10) Pub. No.: US 2022/0251143 A1
(43) Pub. Date: Aug. 11, 2022

(54) NON-HYDROLYZABLE NON-CLEAVABLE, STABLE LINKERS FOR PRECISION THERAPEUTICS AND USES THEREOF

(71) Applicant: THE TRUSTEES OF COLUMBIA UNIVERSITY IN THE CITY OF NEW YORK, New York, NY (US)

(72) Inventors: Antonio Tito Fojo, New York, NY (US); Shi-Xian Deng, White Plains, NY (US); Donald W. Landry, New York, NY (US); Susan E. Bates, New York, NY (US); Xiaoming Xu, Fair Lawn, NJ (US)

(21) Appl. No.: 17/476,656

(22) Filed: Sep. 16, 2021

Related U.S. Application Data
(63) Continuation of application No. PCT/US2020/023285, filed on Mar. 18, 2020.
(60) Provisional application No. 62/819,776, filed on Mar. 18, 2019.

(51) Int. Cl. C07K 7/64; A61K 38/12; A61K 45/06

(52) U.S. Cl. CPC C07K 7/64; A61K 45/06; A61K 38/12

(57) ABSTRACT

The present disclosure provides conjugate compositions comprising non-hydrolyzable, non-cleavable, stable linkers, a targeting moiety, and a therapeutic or chemotherapeutic agent. More specifically the present disclosure provides for compositions comprising non-hydrolyzable, non-cleavable, stable linkers, a somatostatin receptor (SSTR) targeting moiety, such as lanreotide, and a chemotherapeutic agent targeting microtubules, such as mertansine. Methods of using the compositions to treat cancer and other diseases are also provided.

### CROSS-REFERENCE TO OTHER APPLICATIONS

The present application is a continuation of PCT application serial No. PCT/US2020/023285, filed Mar. 18, 2020, which claims priority to U.S. patent application Ser. No. 62/819,776 filed Mar. 18, 2019, all of which are incorporated by reference, as if expressly set forth in their respective entireties herein.

### SEQUENCE LISTING

The instant application contains a Sequence Listing which has been submitted electronically in ASCII format and is hereby incorporated by reference in its entirety. Said ASCII copy, created on Oct. 4, 2021, is named LINKER.txt and is 11.9 kilobytes in size.

### FIELD

The present disclosure relates to targeted delivery of a chemotherapeutic or a therapeutic agent using a targeting moiety. More specifically, the disclosure relates to compositions comprising drug conjugates and related methods of using the compositions for treatment, wherein the compositions comprise non-hydrolyzable, non-cleavable, stable linkers as a portion of the conjugate composition.

### BACKGROUND

Off-target effects of chemotherapeutic drugs, which may result in side effects, often limit current treatments of cancers of all types.

While some drug conjugates have been made and used for targeting and delivering a chemotherapeutic drug to a target cancer cell, until now, it was thought that in order to optimally deliver a drug to a target, a labile linker that ligates the therapeutic agent or drug or payload to the targeting moiety was needed so as to release the drug once the conjugate was delivered to the intracellular compartment. However, this has resulted in the conjugate undergoing hydrolysis in the circulation and the drug being released prior to reaching the target cell or undergoing hydrolysis in the cell and eventually coming out of the cell. This in turn resulted in greater toxicity and the need to reduce the dose of the conjugate and in turn lowered the efficacy of the conjugate and more specifically the drug portion of the conjugate, given its administered dose had to be reduced.

One type of cancer that would benefit from these conjugate compositions is non-Hodgkin lymphoma (NHL). The number of patients with NHL continues to increase. This year, an estimated 72,240 people (40,080 men and 32,160 women) in the United States will be diagnosed with NHL. The disease accounts for 4% of all cancers in the United States. While considered a very treatable cancer, today almost one-third of all patients with this diagnosis still will die from their disease, explaining the American Cancer Society estimate of 20,140 deaths from non-Hodgkin lymphoma in 2017. The overall 5-year survival rate for people with NHL is 69%. The overall 10-year survival rate is 59%. The cornerstone of cancer therapy remains chemotherapy and an important component of all these regimens are agents that target the microtubules which are structures that are important in cell division and more importantly in trafficking inside the cell where they are critical components of the highways on which countless proteins traffic. Thus, this type of cancer would greatly benefit from a precise targeted treatment.

An additional type of cancer that would benefit from a targeted treatment is neuroendocrine prostate cancer. While prostate adenocarcinoma is fairly treatable and curable, neuroendocrine prostate cancer is very aggressive with an expected survival of less than 2 years.

Thus, there is an urgent ongoing need for new therapeutics that specifically target lymphoma cells and neuroendocrine prostate cancer cells, as well as other cancer cells, and limit toxicity to non-tumor cells as well as to increase efficacy. As shown herein, this is achieved with a conjugate composition containing non-hydrolyzable, non-cleavable, stable linker as a portion of the conjugate composition.

### SUMMARY

The present disclosure relates to a chemotherapeutic drug conjugate that specifically targets cancer cells by linking the drug, with unique linkers, to a compound that binds specifically to receptors commonly found on the surface of cancer cells or that can be transiently made to appear on the surface of the cancer cells using agents generally known as epigenetic agents, so as to be able to deliver a toxic payload. This technology and associated strategy allow specific targeting of cancer cells while limiting toxicity to non-tumor cells, thereby reducing and/or eliminating off-target side effects. The unique linkers are non-hydrolyzable and non-cleavable and stable meaning that the entire conjugate composition is delivered to the target cancer cell.

The present disclosure relates to a composition, and a method for treating lymphoma, neuroendocrine prostate cancer, as well as other cancers and diseases in a subject in need thereof comprising administering a therapeutically effective amount of the composition. While malignant lymphomas and neuroendocrine prostate cancer are two prime examples that can be treated with the conjugate compositions described herein, in fact these conjugates can be administered to a diverse group of cancers that either possess the cell surface receptor to which the conjugate attaches or that can have the amount of this cell surface receptor increased or induced, even if transiently. In the case of the diverse group of cancers that may be treated with this conjugate, the administration can then be considered to be tissue agnostic.

In some embodiments, the composition comprises a lanreotide-mertansine (DM1) conjugate with a non-hydrolyzable, non-cleavable, stable linker.

In certain embodiments, the composition comprises lanreotide, mertansine, and N-γ-maleimidobutyryl-oxysuccinimide ester (GMBS), and is denoted DM1-GMBS-Lanreotide or P182-1-DM1, and has the following structure:

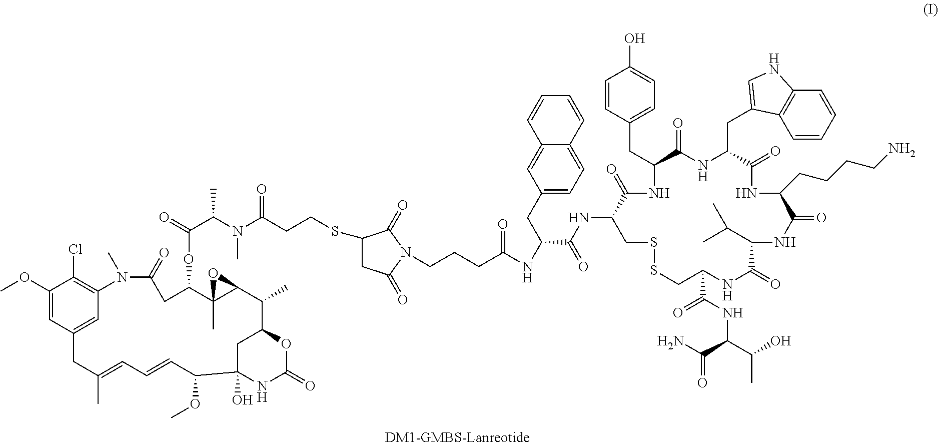 [SMILES: COc1cc2cc(c1Cl)N(C)C(=O)C[C@H](OC(=O)[C@H](C)N(C)C(=O)CCSC1CC(=O)N(CCCC(=O)N[C@H](Cc3ccc4ccccc4c3)C(=O)N[C@H]3CSSC[C@H](C(=O)N[C@H](C(N)=O)[C@@H](C)O)NC(=O)[C@H](C(C)C)NC(=O)[C@H](CCCCN)NC(=O)[C@@H](Cc4c[nH]c5ccccc45)NC(=O)[C@H](Cc4ccc(O)cc4)NC3=O)C1=O)[C@]1(C)O[C@H]1[C@H](C)[C@@H]1C[C@@](O)(NC(=O)O1)[C@H](OC)/C=C/C=C(\C)C2]

In a further embodiment, the composition comprises lanreotide, mertansine, and polyethylene glycol (PEG), and is denoted DM1-PEG-Lanreotide or P182-2-DM1, and has the following structure:

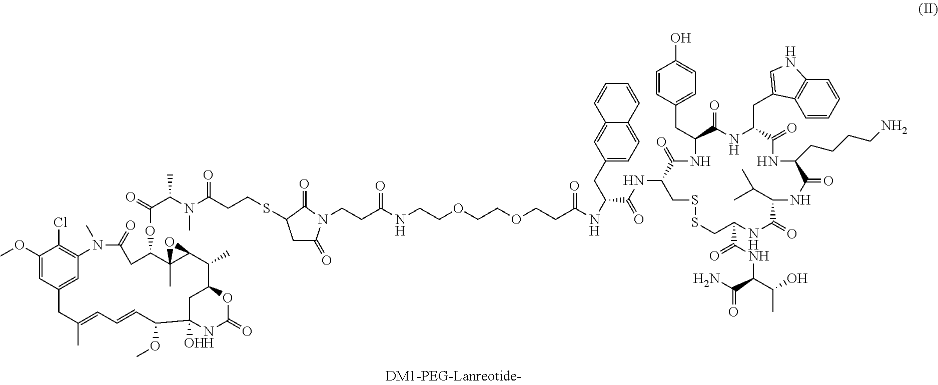 [SMILES: COc1cc2cc(c1Cl)N(C)C(=O)C[C@H](OC(=O)[C@H](C)N(C)C(=O)CCSC1CC(=O)N(CCC(=O)NCCOCCOCCC(=O)N[C@H](Cc3ccc4ccccc4c3)C(=O)N[C@H]3CSSC[C@H](C(=O)N[C@H](C(N)=O)[C@@H](C)O)NC(=O)[C@H](C(C)C)NC(=O)[C@H](CCCCN)NC(=O)[C@@H](Cc4c[nH]c5ccccc45)NC(=O)[C@H](Cc4ccc(O)cc4)NC3=O)C1=O)[C@]1(C)O[C@H]1[C@H](C)[C@@H]1C[C@@](O)(NC(=O)O1)[C@H](OC)/C=C/C=C(\C)C2]

In further embodiments, the composition comprises lanreotide, mertansine, and a non-hydrolyzable, non-cleavable, stable linker, wherein the linker has the structure (A), (B), or (C) and wherein n is an integer of 1 or more.

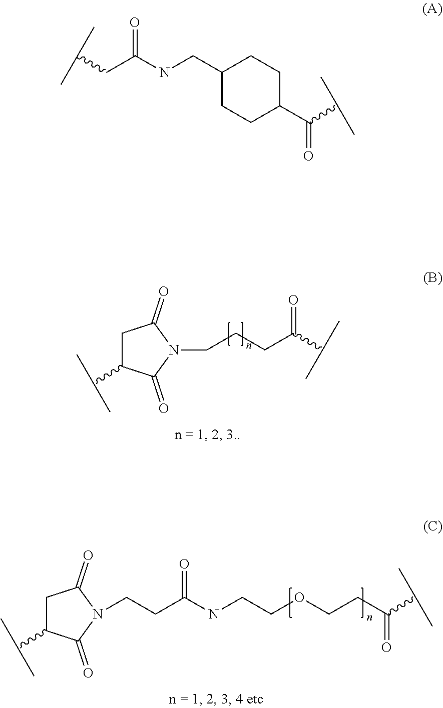

In some embodiments, the composition has one of the following structures wherein n is an integer of 1 or more.

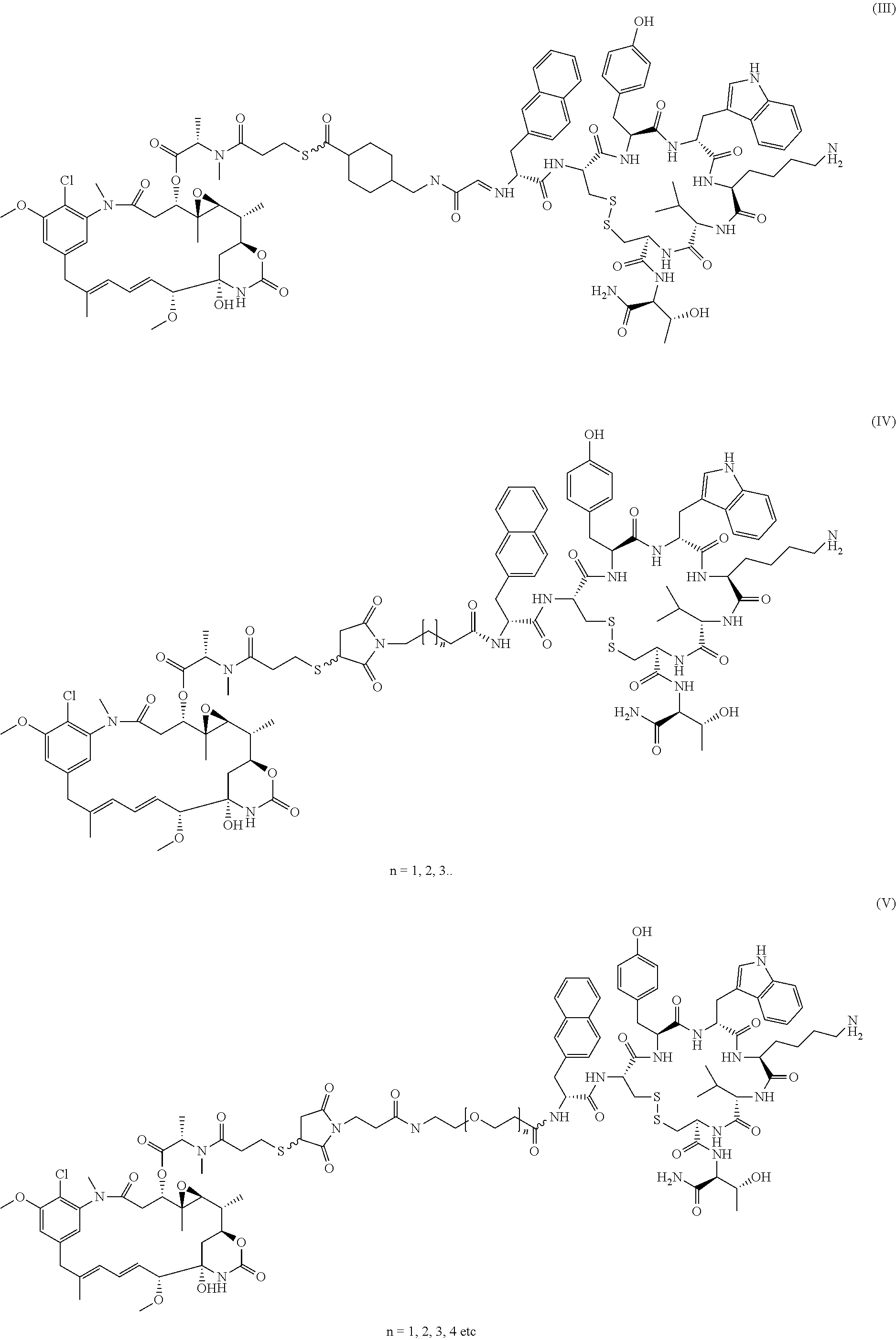

In additional embodiments, the composition comprises a targeting moiety, a chemotherapeutic agent, and GMBS-based linker.

In further embodiments, the composition comprises a targeting moiety, a chemotherapeutic agent, and PEG-based linker.

In still further embodiments, the composition comprises a targeting moiety, a therapeutic agent, and GMBS-based linker.

In further embodiments, the composition comprises a targeting moiety, a therapeutic agent, and PEG-based linker.

In additional embodiments, the composition comprises a targeting moiety, a chemotherapeutic agent, and a non-hydrolyzable, non-cleavable, stable linker having structures including but not limited to (A), (B), and (C).

In additional embodiments, the composition comprises a targeting moiety, a therapeutic agent, and a non-hydrolyzable, non-cleavable, stable linker having structures including but not limited to (A), (B), and (C).

In some embodiments, the composition comprises a somatostatin receptor (SSTR) targeting moiety, a therapeutic or chemotherapeutic agent, and a non-hydrolyzable, non-cleavable, stable linker.

In certain embodiments, the SSTR-targeting moiety targets SSTR1, SSTR2, SSTR3, SSTR4 and/or SSTR5. In certain embodiments, the SSTR-targeting moiety targets SSTR2. In additional embodiments, the SSTR-targeting moiety is a somatostatin analogue. In certain embodiments, the SSTR-targeting moiety is a SSTR agonist. In further embodiments, the SSTR-targeting moiety is a peptide.

In certain embodiments, the SSTR-targeting moiety is selected from the group consisting of lanreotide, octreotide, octreotate, pasireotide, vapreotide, seglitide, and derivatives thereof. In certain embodiments, the SSTR-targeting moiety is radiolabeled.

In certain embodiments, the linkers are one or more chosen from the group of GMBS, PEG and compound structures (A), (B), and (C).

In certain embodiments, the chemotherapeutic agent targets microtubules.

In certain embodiments, the chemotherapeutic agent is a microtubule-destabilizing drug or a microtubule-stabilizing drug.

In yet additional embodiments, the chemotherapeutic agent is mertansine (DM1), DM4, maytansine, or an analog, derivative, prodrug, or pharmaceutically acceptable salt thereof.

In yet additional embodiments, the chemotherapeutic agent is auristatin E [MMAE].

In yet additional embodiments, the chemotherapeutic agent is SN-38, the active metabolite of the chemotherapy drug irinotecan.

In certain embodiments, the compositions can comprise one or more linkers, one or more targeting moieties, and one or more therapeutic or chemotherapeutic agents.

The present disclosure also includes methods of using the disclosed compositions to treat disease including cancer.

In some embodiments, the method further comprises treating the patient with surgery, radiation, and/or a therapeutic agent.

In some embodiments, the method further comprises treating the patient with an agent which induces or increases the expression of a receptor on the target cells. In some embodiments, the receptor is the somatostatin receptor. In some embodiments, the receptor is SSTR2. The agent which increases the levels of the receptor on the surface of the cancer cell, even if transiently, usually belongs to a class of drug commonly referred to as epigenetic modifiers. In some embodiments, the agent is an epigenetic modifier or a combination of epigenetic modifiers. In some embodiments, the agent is a DNA methylation inhibitor including but not limited to 5-aza-2′ deoxycytidine. In some embodiments, the agent is a histone deacetylase inhibitor including but not limited to trichostatin A, romidepsin also known as Istodax, belinostat, entinostat, panobinostat and vorinostat, as well as other similar agents that can increase the receptor levels by increasing histone acetylation. In some embodiments the agent is a histone methyltransferase inhibitor, including but not limited to tazemetostat or pinometostat. In some embodiments the agent is an IDH1/2 inhibitor including but not limited to ivosidenib and enasidenib. In some embodiments the agent is a histone acetyltransferase inhibitor. In some embodiments, the agent is a histone demethylase inhibitor including but not limited to GSK2879552 or tranylcypromine. In some embodiments, the agent is a bromodomain and extraterminal domain (BET) protein inhibitor including but not limited to molibresib. Other epigenetic modifiers are in development and can also be envisioned to be useful in future embodiments, as epigenetic modifiers change gene expression.

In some embodiments, the cancer is lymphoma. In some embodiments, the lymphoma is non-Hodgkin lymphoma (NHL).

In some embodiments, the cancer is neuroendocrine prostate cancer.

In some embodiments, the cancer is sarcoma, neuroblastoma, glioblastoma, melanoma, lung carcinoma, non-small cell lung cancer, glioma, head and neck cancer, prostate cancer other than neuroendocrine prostate cancer, colorectal cancer, liver cancer, ovarian cancer, pancreatic cancer, squamous cell cancer, mesothelioma, breast cancer, brain cancer, cervical cancer, stomach cancer, and leukemia as well as neuroendocrine tumors or carcinoid tumors.

In some embodiments, the compositions and therapeutic methods described herein can be used for the treatment of additional diseases including but not limited to heart disease and brain diseases such as Alzheimer's disease.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1 is a bar graph showing the cytotoxicity of a lanreotide-DM1 conjugate composition, DM1-GMBS-Lanreotide or P182-1-DM1, in Pffifer cells after 72 hours of treatment.

FIG. 2 is a bar graph showing the cytotoxicity of a lanreotide-DM1 conjugate composition, DM1-GMBS-Lanreotide or P182-1-DM1, in Karpas cells after 72 hours of treatment.

FIG. 3 is a bar graph showing the cytotoxicity of a lanreotide-DM1 conjugate composition, DM1-PEG-Lanreotide or P182-2-DM1, in Pffifer cells after 72 hours of treatment.

FIG. 4 is a bar graph showing the cytotoxicity of a lanreotide-DM1 conjugate composition, DM1-PEG-Lanreotide or P182-2-DM1, in Karpas cells after 72 hours of treatment.

FIG. 5 is a bar graph showing the cytotoxicity of a lanreotide-DM1 conjugate composition, DM1-GMBS-Lanreotide or P182-1-DM1, in Pffifer cells after 96 hours of treatment.

FIG. 6 is a bar graph showing the cytotoxicity of a lanreotide-DM1 conjugate composition, DM1-GMBS-Lanreotide or P182-1-DM1, in Karpas cells after 96 hours of treatment.

FIG. 7 is a bar graph showing the cytotoxicity of a lanreotide-DM1 conjugate composition, DM1-PEG-Lanreotide or P182-2-DM1, in Pffifer cells after 96 hours of treatment.

FIG. 8 is a bar graph showing the cytotoxicity of a lanreotide-DM1 conjugate composition, DM1-PEG-Lanreotide or P182-2-DM1, in Karpas cells after 96 hours of treatment.

FIG. 9 is a blot showing the expression of SSTR2 in malignant lymphoma cells Karpas and Pffifer as compared to NET cells.

FIG. 10 is a bar graph showing cytotoxicity of a DM1-lanreotide conjugate composition in malignant lymphoma cells.

FIG. 11 is a bar graph showing fold increase of expression of SSTR2 in neuroendocrine cancer cell lines [NEC1, NEC, NEC3] treated with two epigenetic agents [EA1, EA2] within 72 hours at concentrations listed in the X-axis. FIG. 11A shows the results for cell line NEC1.

FIG. 11B shows the results for cell line NEC2. FIG. 11C shows the results for cell line NEC3.

FIG. 12 is an immunoblot showing expression of SSTR2 in brain lysate, neuroendocrine prostate cancer (NEPC) organoid, and Pffifer cells.

FIG. 13 are dose response curves for NEPC organoids treated for three days with DM1, Lan-MCC-DM1 and 182-2-DM1 at concentrations of 1 uM, 0.33 uM, 0.11 uM, 0.037 uM, 0.012 uM, 0.0041 uM, 0.00137 uM and 0.00046 uM. FIG. 13A shows the dose response curve of DM1.

FIG. 13B shows the dose response curve of conjugate composition, lanreotide-MCC-DM1.

FIG. 13C shows the dose response curve of conjugate composition 182-2-DM1 (lanreotide-PEG-DM1).

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means an animal with an immune system such as avians and mammals. Mammals include canines, felines, rodents, bovine, equines, porcines, ovines, and primates. Avians include, but are not limited to, fowls, songbirds, and raptors. Thus, the compositions and methods described herein can be used in veterinary medicine, e.g., to treat companion animals, farm animals, laboratory animals in zoological parks, and animals in the wild. They are particularly desirable for human medical applications.

The term “patient” as used in this application means a human subject. In some embodiments, the patient is suffering with cancer. In some embodiments, the patient is suffering with lymphoma. In some embodiments, the patient is suffering with non-Hodgkin lymphoma. In some embodiments, the patient is suffering with neuroendocrine prostate cancer. In some embodiments the patient is suffering with prostate cancer other than neuroendocrine prostate cancer.

In other embodiments the patient is suffering from other cancers, these being of different types. In some embodiments, the cancer cells are expressing one of the somatostatin receptors to which the targeting moiety of the conjugate can bind so as to deliver the chemotherapeutic agent, i.e., chemotherapeutic drug or payload, to an intracellular location. In other embodiments the patient's cancer does not express the somatostatin receptor but expression has been induced, even if transiently, with one or more epigenetic agents as these agents can induce or increase the expression of the somatostatin receptor.

The term “lymphoma” as used herein is a cancer of lymphatic cells of the immune system. Lymphomas typically present as a solid tumor. Exemplary lymphomas include small lymphocytic lymphoma, lymphoplasmacytic B-cell lymphoma, Waldenström macroglobulinemia, splenic marginal zone lymphoma, plasmacytoma, extranodal marginal zone B cell lymphoma, MALT lymphoma, nodal marginal zone B cell lymphoma (NMZL), follicular lymphoma, mantle cell lymphoma, diffuse large B cell lymphoma (DLBCL), mediastinal (thymic) large B cell lymphoma, intravascular large B cell lymphoma, primary effusion lymphoma, Burkitt lymphoma, chronic lymphocytic lymphoma, adult T cell lymphoma, nasal type extranodal NK/T cell lymphoma, enteropathy-type T cell lymphoma, hepatosplenic T cell lymphoma, blastic NK cell lymphoma, mycosis fungoides, Sezary syndrome, primary cutaneous CD30-positive T cell lymphoproliferative disorders, primary cutaneous anaplastic large cell lymphoma, lymphomatoid papulosis, angioimmunoblastic T cell lymphoma, unspecified peripheral T cell lymphoma, and anaplastic large cell lymphoma.

The term “agent” as used herein means a substance that produces or is capable of producing an effect and would include, but is not limited to, chemicals, pharmaceuticals, biologics, small organic molecules, antibodies, nucleic acids, peptides, and proteins.

As used herein, the terms “reduce or inhibit” refer to the ability to cause an overall decrease of 10%, 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

The terms “treat”, “treatment”, and the like refer to a means to slow down, relieve, ameliorate or alleviate at least one of the symptoms of the disease, or reverse the disease after its onset.

As used herein, the phrases “treating cancer” and “treatment of cancer” and “treatment of tumors” mean to decrease, reduce, or inhibit the replication or the growth of cancer cells; decrease, reduce or inhibit the spread (formation of metastases) of cancer; decrease tumor size or inhibit its growth or prevent its growth or retard its growth; decrease the number of tumors (i.e., reduce tumor burden); lessen or reduce the number of cancerous cells in the body; prevent recurrence of cancer after surgical removal or other anti-cancer therapies; or ameliorate or alleviate the symptoms of the disease caused by the cancer.

As used herein, the term “therapeutically effective” means that the amount of the composition used is of sufficient quantity to ameliorate one or more causes or symptoms of a disease or disorder. Such amelioration only requires a reduction or alteration, not necessarily elimination. A “therapeutically effective amount” will vary depending on the agent, the disorder and its severity and the age, weight, physical condition and responsiveness of the subject to be treated.

The term “in need thereof” would be a subject known or suspected of having or being at risk of developing lymphoma or another type of cancer or disease.

A “non-cleavable” linker, as used herein, refers to any linker that cannot be cleaved physically, chemically or enzymatically. Examples for physical cleavage may be cleavage by light, radioactive emission or heat, while examples for chemical cleavage include cleavage by redox reactions, hydrolysis, or pH-dependent cleavage. Cleavage by enzymes involves the cleavage by proteins whose function is to cleave covalent bonds.

A “non-hydrolyzable linker” as used herein refers to any linker that cannot be hydrolyzed. Because the linker cannot be hydrolyzed it can be said to be stable.

Because the linker cannot be hydrolyzed or cleaved, then of necessity the entire conjugate represents the active drug and can engage its target and have the desired effect—in the case of a cancer cell to arrest its growth or to cause its destruction. In effect the active agent is the entire conjugate. The accessibility of the site or region of a molecule or a macromolecule to which the chemotherapy agent binds is such that the active chemotherapy agent can bind despite it being permanently attached to its linker and the portion of the molecule that was important in targeting it to the cancer cell, i.e., the targeting moiety.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

### Abbreviations

CPP Cell penetrating peptide
TTP tumor targeting peptides
MTA microtubule-targeting agent
DM1 mertansine
SSTR somatostatin receptor
PEG polyethylene glycol
GMBS N-γ-maleimidobutyryl-oxysuccinimide ester
NHL non-Hodgkin lymphoma
NET neuroendocrine tumor
NEPC neuroendocrine prostate cancer

Described herein is a composition which includes a molecule designed to specifically target lymphoma cells in non-Hodgkin lymphoma (NHL), neuroendocrine prostate cancer and other cancers, including prostate cancer other than neuroendocrine prostate cancer. The target can also be cancer cells in which the expression of the somatostatin receptor can be induced, even if transiently, by either a single or a combination of epigenetic agents, and deliver a chemotherapeutic agent, drug or payload to treat the disease. The target can also be cancer cells in which the expression of a receptor expressed on cancer cells can be induced, even if transiently, by either a single or a combination of epigenetic agents, and deliver a chemotherapeutic agent, drug or payload to treat the disease.

NHL tumor cells have been shown to express the somatostatin receptor (SSTR) on their surface. The conjugate composition comprises an agent, drug or payload which may be mertansine (DM1), a microtubule targeting chemotherapeutic agent, conjugated to lanreotide, a somatostatin analogue. The lanreotide aspect of this drug can specifically bind to SSTR on the cell surface of lymphoma cells so that the cytotoxicity of the mertansine aspect can kill lymphoma cells specifically. This limits the molecule's toxicity on cells that do not express the SSTR on their surface, in the case cells that are not lymphoma cells, thus reducing the side effects that are typically associated with microtubule targeting chemotherapeutic agents.

Neuroendocrine prostate cancer cells have also been shown to express SSTR on their surface. Additionally, prostate cancer cells in patients who have experienced progression of their prostate cancer after treatment with an anti-hormonal agent also express high levels of functional somatostatin receptor. Note that this is prostate cancer that is not defined or thought to be neuroendocrine prostate cancer, but simply prostate cancer. The SSTR levels of the cancer appear and rise as the patient's cancer progresses to frank neuroendocrine prostate cancer. Thus, patients in this precursor state which has not yet been clinically classified as neuroendocrine prostate cancer can also benefit from a composition targeting SSTR.

The compositions further comprise at least one non-hydrolyzable, non-cleavable, stable linker.

In certain embodiments, the composition is used in targeted treatment for non-Hodgkin lymphoma (NHL). In certain embodiments, the composition is used in targeted treatment for neuroendocrine prostate cancer and the precursor state of neuroendocrine prostate cancer. In certain embodiments, the composition is used in treating cancer cells expressing SSTR or treating SSTR+ cancer. In certain embodiments, the composition is used in treating cancer cells expressing SSTR2 or treating SSTR2+ cancer.

It is expected that the DM1-Lanreotide conjugate and similar compositions described herein using a non-hydrolyzable, non-cleavable, stable linker will deliver to malignant lymphomas and other cancers expressing the somatostatin receptor and other tumor specific receptors, or cancer cells in which the expression of these receptors can be induced or increased, even if transiently, a highly potent chemotherapeutic agent, e.g., microtubule-targeting agent (MTA). The conjugate will be more potent and more specific than the chemotherapeutic agent or drug (e.g., a MTA) alone. The delivery in a precise manner will abrogate neurotoxicity and bone marrow suppression, common problems that often lead to dose reductions or discontinuation and that have also led to the routine capping of vincristine and other potent chemotherapeutic drug doses in other settings. The conjugate will allow the potential benefit from MTAs to be leveraged to its maximum. The conjugate using the non-hydrolyzable, non-cleavable, stable linker also will allow even more potent MTAs to be used because the MTAs will not be released in the blood or other tissue other than inside the cancer cells that comprise the tumor tissue. This will alleviate the potential side effects of the more potent MTAs. Additionally, because the linker is not cleaved or hydrolyzed, then the therapeutic active agent (also known as the payload or the chemotherapeutic agent) is never released and cannot leave the cell as a free active compound. By preventing this from happening, free therapeutic active agent is prevented from leaving the cell and going elsewhere in the body. By preventing this movement of free therapeutic agent or payload or chemotherapeutic agent, its entry into all other cells is prevented unless the cell has on its surface the appropriate receptor. This further prevents side effects from occurring.

In certain embodiments, the composition comprises a single targeting moiety and a single chemotherapeutic or therapeutic agent. In certain embodiments, the composition contains one or more targeting moieties, optionally one or more non-hydrolyzable, non-cleavable, stable linkers, one or more chemotherapeutic agents, or therapeutic agents, or any combination thereof. The composition can have any number of targeting moieties, non-hydrolyzable, non-cleavable, stable linkers, and chemotherapeutic agents or therapeutic agents. In certain embodiments, the composition can contain more than one type of targeting moieties, more than one type of a non-hydrolyzable, non-cleavable, stable linker, and/or more than one type of chemotherapeutic agent or therapeutic agent. In certain embodiments, the composition can contain more than one targeting moiety attached to a single chemotherapeutic agent or therapeutic agent. In certain embodiments, the composition can contain more than one chemotherapeutic agent or therapeutic agent attached to a single targeting moiety.

In some embodiments, the targeting moiety binds to the non-hydrolyzable, non-cleavable, stable linker at the C-terminus. In some embodiments, the targeting moiety binds to the non-hydrolyzable, non-cleavable, stable linker at the N-terminus.

The linkers are non-hydrolyzable and non-cleavable and thus are stable. At least five linker compound structures, GMBS, PEG, and structures (A), (B), and (C) shown herein meet this requirement and can be incorporated into the conjugate structures, including those shown as (I)-(V).

In certain embodiments, the molar ratio of the targeting moiety to the chemotherapeutic or therapeutic agent in the composition is about 1:1, 10:1, 9:1, 8:1, 7:1, 6:1, 5:1, 4:1, 3:1, 2:1, 1:2, 1:3, 1:4; 1:5, 1:6, 1:7, 1:8, 1:9, or 1:10.

In certain embodiments, the chemotherapeutic agent or therapeutic agent of the composition comprises a predetermined molar weight percentage from about 1% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the molar weight percentages of the components of the conjugate is 100%.

In some embodiments, the composition comprises a lanreotide-mertansine (DM1) conjugate with a non-hydrolyzable, non-cleavable, stable linker.

In certain embodiments, the composition comprises lanreotide, mertansine, and N-γ-maleimidobutyryl-oxysuccinimide ester (GMBS), and is denoted DM1-GMBS-Lanreotide or P182-1-DM1, and has the following structure:

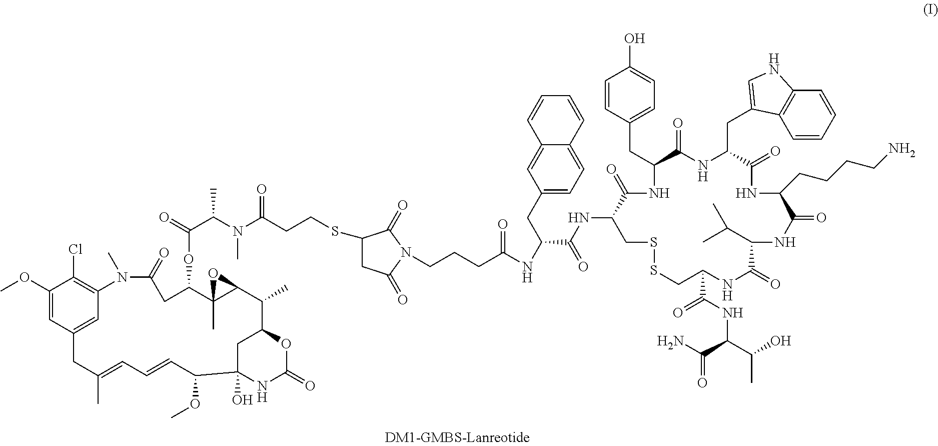 [SMILES: COc1cc2cc(c1Cl)N(C)C(=O)C[C@H](OC(=O)[C@H](C)N(C)C(=O)CCSC1CC(=O)N(CCCC(=O)N[C@H](Cc3ccc4ccccc4c3)C(=O)N[C@H]3CSSC[C@H](C(=O)N[C@H](C(N)=O)[C@@H](C)O)NC(=O)[C@H](C(C)C)NC(=O)[C@H](CCCCN)NC(=O)[C@@H](Cc4c[nH]c5ccccc45)NC(=O)[C@H](Cc4ccc(O)cc4)NC3=O)C1=O)[C@]1(C)O[C@H]1[C@H](C)[C@@H]1C[C@@](O)(NC(=O)O1)[C@H](OC)/C=C/C=C(\C)C2]

In a further embodiment, the composition comprises lanreotide, mertansine, and polyethylene glycol (PEG), and is denoted DM1-PEG-Lanreotide or P182-2-DM1, and has the following structure:

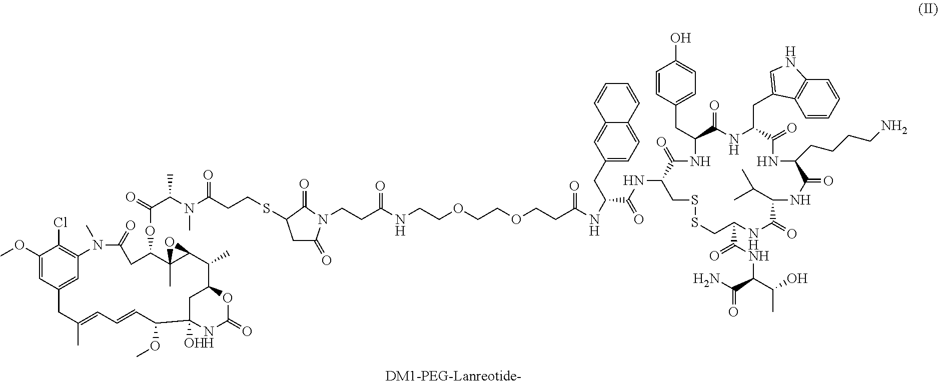 [SMILES: COc1cc2cc(c1Cl)N(C)C(=O)C[C@H](OC(=O)[C@H](C)N(C)C(=O)CCSC1CC(=O)N(CCC(=O)NCCOCCOCCC(=O)N[C@H](Cc3ccc4ccccc4c3)C(=O)N[C@H]3CSSC[C@H](C(=O)N[C@H](C(N)=O)[C@@H](C)O)NC(=O)[C@H](C(C)C)NC(=O)[C@H](CCCCN)NC(=O)[C@@H](Cc4c[nH]c5ccccc45)NC(=O)[C@H](Cc4ccc(O)cc4)NC3=O)C1=O)[C@]1(C)O[C@H]1[C@H](C)[C@@H]1C[C@@](O)(NC(=O)O1)[C@H](OC)/C=C/C=C(\C)C2]

In further embodiments, the composition comprises lanreotide, mertansine, and a non-hydrolyzable, non-cleavable, stable linker, wherein the linker has the structure (A), (B), or (C) and wherein n is an integer of 1 or more.

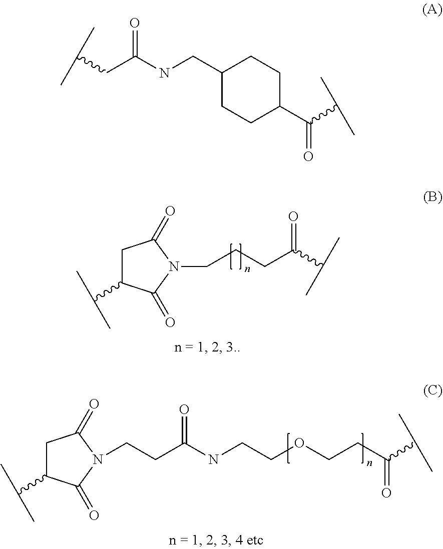

In some embodiments, the composition has one of the following structures wherein n is an integer of 1 or more.

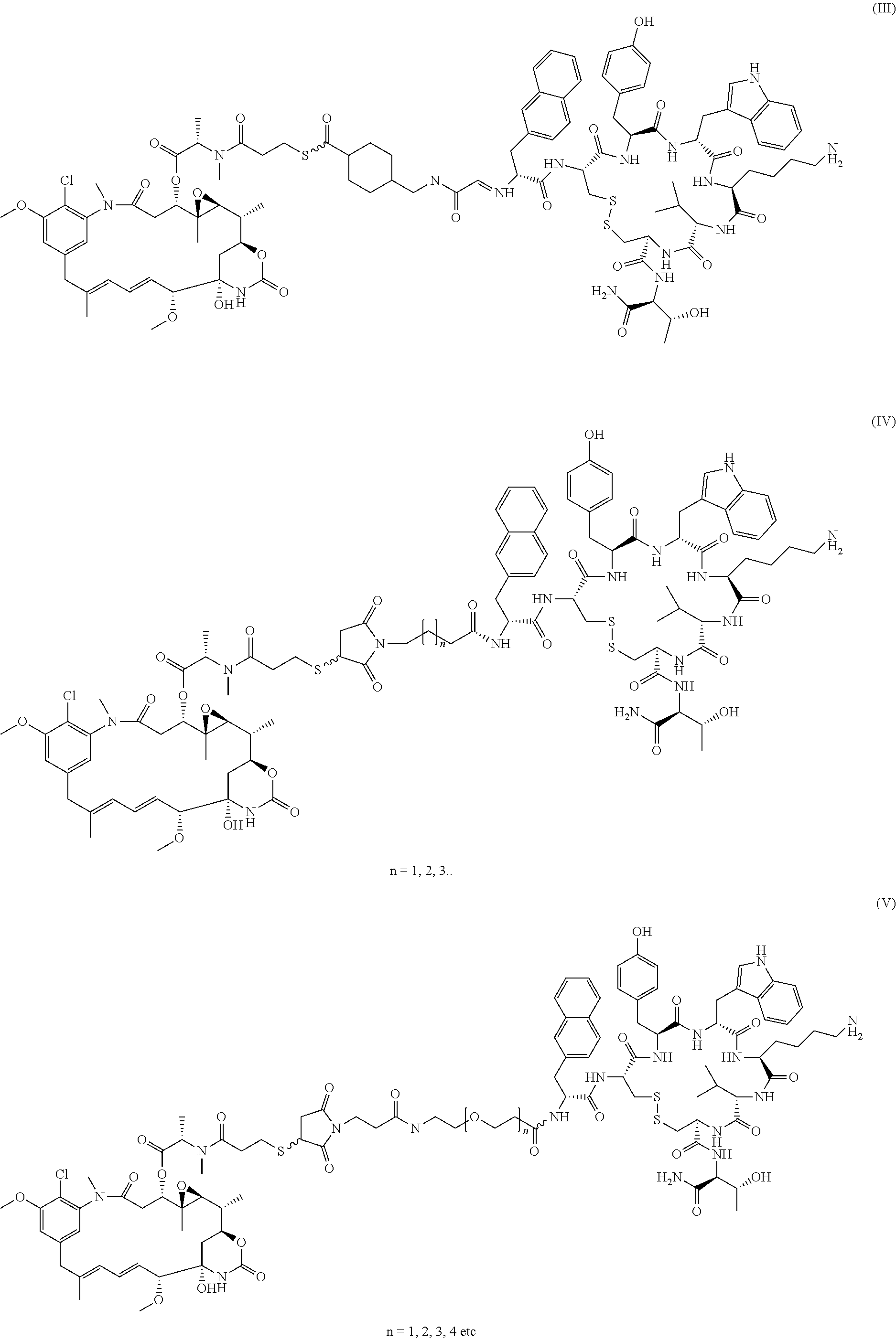

In additional embodiments, the composition comprises a targeting moiety, a chemotherapeutic agent, and GMBS-based linker.

In further embodiments, the composition comprises a targeting moiety, a chemotherapeutic agent, and PEG-based linker.

In still further embodiments, the composition comprises a targeting moiety, a therapeutic agent, and GMBS-based linker.

In further embodiments, the composition comprises a targeting moiety, a therapeutic agent, and PEG-based linker.

In additional embodiments, the composition comprises a targeting moiety, a chemotherapeutic agent, and a non-hydrolyzable, non-cleavable, stable linker having structures including but not limited to (A), (B), and (C).

In additional embodiments, the composition comprises a targeting moiety, a therapeutic agent, and a non-hydrolyzable, non-cleavable, stable linker having structures including but not limited to (A), (B), and (C).

### Targeting Moieties

A targeting moiety as used herein is an agent or moiety which targets a particular receptor or protein or other cell surface molecule or macromolecule in order to deliver a therapeutic or chemotherapeutic agent, i.e., payload. In some embodiments, the targeting moiety is a small molecule. In some embodiments, the targeting moiety is a peptide. In some embodiments, the receptor or protein is associated with a disease such as cancer or heart disease.

Any molecule or peptide small enough in size such as not to interfere with the therapeutic or chemotherapeutic agent (e.g., DM1) can be used in a conjugate composition with the chemotherapeutic agent and a non-hydrolyzable, non-cleavable, stable linker. Examples of other chemotherapeutic agents that may be used in a conjugate composition include monomethyl auristatin E (MMAE), SN-38, rocaglamide or MZ735 or other inhibitors of eIF4A—chemotherapeutic agents that engage areas on the surface of their targets.

Thus, a further embodiment is a composition comprising a therapeutic or chemotherapeutic agent and a targeting moiety and a non-hydrolyzable, non-cleavable stable linker, wherein the targeting moiety is a peptide or a polypeptide or other small molecules ranging in size from about one amino acid to as many as 40 amino acids, or from a molecular weight of 80 to a molecular weight of about 5000. The peptide, polypeptide or molecule can be linear or cyclic, naturally occurring or synthetic.

Peptides, or polypeptides which specifically target receptors or proteins overexpressed in or related to the surface of cancer cells are sometimes termed tumor targeting peptides (TTPs) or homing peptides. Such targeting can also occur with other small molecules that are not peptides or polypeptides.

One example of a TTP is a peptide containing the motif RGD (Arg-Gly-Asp). This motif is recognized by integrins αvβ(3) and αvβ(5) which are implicated in angiogenesis and overexpressed in tumor cells within many solid tumors including but not limited to sarcomas, neuroblastomas, glioblastomas, melanomas, lung carcinomas, and breast cancer. One such peptide is GSSSGRGDSPA (SEQ ID NO: 1). A further example of an RGD peptide is cilengitide, the salt of a cyclized RGD-based pentapeptide. Cilengitide is undergoing testing in phase I, II and III clinical trials for non-small cell lung cancer, glioma, head and neck cancer, and prostate cancer. Yet another example of an RGD peptide is DNX-2401 in phase I studies for malignant gliomas. Another example is RGD-K5 used for breast cancer. Further examples are LXW7 (cRGDdvc) (SEQ ID NO: 2), cyclo(RGDfK) (SEQ ID NO: 3), and GRGDFSK (SEQ ID NO: 4). RGD peptides are also being made that include a tissue penetration motif, R/KXXR/K (SEQ ID NO: 5).

The motif RTD is recognized by the integrin αvβ(6) which is overexpressed on the surface of tumor cells including but not limited to colon, liver, ovarian, pancreatic, and squamous cell cancers.

A further example of a TTP is a peptide containing the motif NGR which recognizes and binds to aminopeptidase N (also known as CD13) that is overexpressed by endothelial cells of many tumors.

Both NGR and RGD peptides have been used to deliver TNF. NGR peptides are in clinical trials for ovarian, lung, colon, hepatocellular carcinoma, sarcoma, and mesothelioma.

Further examples of TTPs are TCP-1 and F56 targeting colorectal cancer.

Further examples of TTPs include but are not limited to peptides that target and bind to peptide transporter 1 (PEPT1), EGFR, HER2, PSMA, MUC1, Upar, GRPR, SSTRs, CCKRs, NTR1, TfRs, VEGFR, Insulin, and erphrin receptors. See Table 1 for the receptor, the peptide sequence, and the cancer to which the peptide targets.

TABLE 1
Targeting Peptides

| Receptor | Peptide Sequence | Tumor Type |
|---|---|---|
| αvβ3 | Cilengitide | Lung, prostate, glioblastoma |
| αvβ3 | LXW7 (cGRGDdvc) (SEQ ID | Glioblastoma/melanoma |
| | NO: 2) | |
| αvβ3 | Cyclo (RGD-D-FL) SEQ ID | Breast |
| | NO: 6) | |
| αvβ3 | Ac-GRGDFSL-OH (SEQ ID | ND |
| | NO: 7) | |
| αvβ6 | RTDLXXL (SEQ ID NO: 8) | Pancreatic |
| APN | NGR | Colorectal, ovarian, lung, |
| | | hepatocellular carcinoma |
| PEPT1 | Ser-Glu | Pancreatic, cervical |
| EGFR | GE11 (YHWYGYTPQNVI) | Hepatoma |
| | (SEQ ID NO: 9) | |
| EGFR | EHGAMEI (SEQ ID NO: 10) | Hepatoma |
| EGFR | DE (LARLLT) (SEQ ID NO: | NSCLC |
| | 11) | |
| EGFR | EDA (Pg-YNPTTYQ-Aha) | Breast |
| | (SEQ ID NO: 12) | |
| EGFR | Disruptin (SVDNPH) (SEQ ID | HNSCC, lung |
| | NO: 13) | |
| HER2 | KCCYSL (SEQ ID NO: 14) | Ovarian |
| HER2 | LTVSPWY (SEQ ID NO: 15) | ND |
| PSMA | KYLAYPDSVHIW (SEQ ID | Prostate |
| | NO: 16) | |
| PSMA | WQPDTAHHWATL (SEQ ID | Prostate |
| | NO: 17) | |
| MUC1 | GO-201 | Breast, prostate |
| uPAR | AE105 (D-Cha-F-s-r-Y-L-W- | Glioma |
| | S) (SEQ ID NO: 18) | |
| | VSNKYFSNIHW (SEQ ID | Prostate |
| | NO: 19) | |
| GRPR | EQRLGNQWAVGHLM | Prostate |
| | (SEQ ID NO: 20) | |
| GRPR | QWAVGHLM (SEQ ID NO: | Prostate |
| | 21) | |
| SSTRs | OCT (FCFWKTCT) (SEQ ID | Pancreatic |
| | NO: 22) | |
| CCKRs | CCK8 (DWMGWMDF) (SEQ | Lung |
| | ID NO: 23) | |
| NTR1 | QLYENKRRPYIL (SEQ ID | SCLC, colorectal, pancreatic, |
| | NO: 24) | prostate |
| | RRPYIL (SEQ ID NO: 25) | SCLC, colorectal, pancreatic, |
| | | prostate |
| TfRs | HAIYPRH (SEQ ID NO: 26) | Leukemia, hepatocellular |
| | | carcinoma |
| VEGFR | CPQPRPLC (SEQ ID NO: 27) | ND |
| VEGFR | K237 (HTMYYHHYQHHL) | Breast |
| | (SEQ ID NO: 28) | |
| VEGFR | ATWLPPR (SEQ ID NO: 29) | ND |
| VEGFR | Peptide SP5.2 | ND |
| | (NGYEIEWYSWVTHGMT) | |
| | (SEQ ID NO: 30) | |
| | | ND |
| Insulin | MCR | ND |
| | (RRLFYKKVGLFYKKVRR) | |
| | (SEQ ID NO: 31) | |
| Ephrin receptors | EWLSPNLAPSVR (SEQ ID | ND |
| | NO: 32) | |
| Ephrin receptors | SNEWIQPRLPQH (SEQ ID | ND |
| | NO: 33) | |
| Ephrin receptors | TNYLFSPNGPIA (SEQ ID | ND |
| | NO: 34) | |
| Ephrin receptors | APY (APYCVYRGSWSC) | ND |
| | (SEQ ID NO: 35) | |
| Ephrin receptors | KYL (KYLPYWPVLSSL) | ND |
| | (SEQ ID NO: 36) | |
| Ephrin receptors | VTM (VTMEAINLAFPG) | ND |
| | (SEQ ID NO: 37) | |
| Ephrin receptors | TYY | ND |
| | [c (CTYYWPLPC)] (SEQ ID | |
| | NO: 38) | |
| Ephrin receptors | YSA peptide | Breast |
| | (YSAYPDSVPMMS) (SEQ | |
| | ID NO: 39) | |
| Ephrin receptors | SWL peptide | Breast |
| | (SWLAYPGAVSYR) (SEQ | |
| | ID NO: 40) | |

ND—not determined at this time

Peptides that target intracellular receptors of cancer can be used in the disclosed compositions. One such example are peptides targeting the BCR/ABL fusion protein that is responsible for the chronic phase of chronic myelogenous leukemia. These peptides are rich in serine and proline include IPTLPSS (SEQ ID NO: 41), YRAPWPP (SEQ ID NO: 42), SSPSTSY (SEQ ID NO: 43) and AHKMGTP (SEQ ID NO: 44).

Peptides that target the extracellular matrix can be used in the disclosed compositions including those that target fibronectin-fibrin complex (CRKEA) (SEQ ID NO: 45).

Peptides can also be cell penetrating peptides (CPPs) that enter the cells directly through membranes or use an endocytotic mechanism to deliver a chemotherapeutic agent.

Cell penetrating peptides (CPPs) allow the payload to be transported through the cell membrane. One example of a CPP is the Tat sequence (GRKKRRQRRPPQ) (SEQ ID NO: 46). A further example is the PFDYLI (SEQ ID NO: 55) peptide. A further example is a pH (low)—dependent Insertion Peptide (pHLIP) which exploits the acidic extracellular environment in cancer.

A further example of a CPP is one that binds to a matrix metalloproteinase (MMPS) which is overexpressed in some tumors. One such peptide is CTX which can cross the blood-brain barrier and penetrate solid tumors. These include AaCtx (MCIPCFTTNPNMAAKCNACCG-SRRGS-CRGPQCIC) (SEQ ID NO: 47) from the venom of the Androctonus australis scorpion, BmKCTa (CGPCFTTDANMARKCRECCG-GI-GK-CFGPQCLCNRI) (SEQ ID NO: 48) from the venom of the Buthus martenzii scorpion, and GaTx1 (CGPCFTTDHQMEQKCAECCG-GI-GK-CYGPQCIC) (SEQ ID NO: 49) and GaTx2 (VSCEDCPDHCSTQKARAKCDNDKCVCEPI) (SEQ ID NO: 50) both from the venom of the Leiurus quinquestriatus scorpion.

See Generally, Zhao et al. 2018; Boohaker et al. 2012; Xiao et al. 2015.

Peptides which target receptors associated with diseases or conditions other than cancer can also be used in the disclosed conjugate compositions. One example of a peptide (DEMEFTEAESNMN) (SEQ ID NO: 51) that targets the G protein-coupled receptor kinase implicated in heart disease and brain diseases such as Alzheimer's disease. See Asai et al. 2014.

Examples of other targeting moieties include the chemokine receptor ligand CXCL12 that binds to the chemokine receptors CXCR4 and CXCR7.

Examples of other targeting moieties that are peptides include monomeric peptides that can bind to PSMA with the sequence WQPDTAHHWATL (SEQ ID NO: 17) and a dimeric version of this peptide, or of similar peptides. PSMA, also known as prostate-specific membrane antigen, is highly expressed by both normal and malignant prostate epithelial cells and by the neovasculature of many tumor types, however, it is not expressed by normal endothelial cells or other normal tissues.

Somatostatin Receptor (SSTR) Targeting Moiety

The targeting moiety in the disclosed compositions may comprise a SSTR-targeting moiety, which may target SSTR1, SSTR2, SSTR3, SSTR4, and/or SSTRS, e.g., human SSTR1, SSTR2, SSTR3, SSTR4, and/or SSTR5. In certain embodiments, the SSTR-targeting moiety targets SSTR2. In certain embodiments, the binding of the conjugate to SSTR2 is stronger than the binding of the conjugate to SSTR1, SSTR3, SSTR4 or SSTR5. In some embodiments, the SSTR-targeting moiety is a somatostatin receptor binding moiety that binds to somatostatin receptors 2 and/or 5.

The SSTR-targeting moiety may be natural or synthetic.

In certain embodiments, the SSTR-targeting moiety is somatostatin or a somatostatin analogue. In some embodiments, the somatostatin analog contains between 8 and 18 amino acids, and includes the core sequence: cyclo[Cys-Phe-D-Trp-Lys-Thr-Cys] (SEQ ID NO: 52) or cyclo[Cys-Tyr-D-Trp-Lys-Thr-Cys] (SEQ ID NO: 53). For example, the C-terminus of the analog is Thr-NH2.

In some embodiments, the SSTR-targeting moiety may be selected from somatostatin, octreotide, lanreotide, Tyr³-octreotate (TATE), vapreotide, cyclo(AA-Tyr-DTrp-Lys-Thr-Phe) (SEQ ID NO: 54) where AA is α-N-Me lysine or N-Me glutamic acid, pasireotide, seglitide, or any other example of somatostatin receptor binding ligands.

In certain embodiments, the SSTR-targeting moiety is a SSTR agonist, e.g., a SSTR2 agonist.

In certain embodiments, the SSTR-targeting moiety is lanreotide or octreotide.

### Chemotherapeutic Agents

A “chemotherapeutic agent” or “chemotherapeutic drug” is a chemical compound useful in the treatment of cancer, regardless of mechanism of action. Classes of chemotherapeutic agents include, but are not limited to, microtubule-targeting agents (MTAs) (or microtubule-target moieties), DNA damaging agents, alkylating agents, antimetabolites, spindle poison plant alkaloids, cytotoxic/antitumor antibiotics, topoisomerase inhibitors, eIF4A inhibitors, antibodies, photosensitizers, and kinase inhibitors.

The chemotherapeutic agent may be natural or synthetic.

In some embodiments, the chemotherapeutic agent is a small molecule.

The chemotherapeutic agent can be an inorganic or organometallic compound containing one or more metal centers. In some examples, the compound contains one metal center. The active agent can be, for example, a platinum compound, a ruthenium compound, cobalt compound, copper compound, or iron compounds.

In certain embodiments, the chemotherapeutic agent is a microtubule-targeting agent (MTA) or tubulin-targeting moiety. In certain embodiments, the chemotherapeutic agent is a microtubule-stabilizing agent or a microtubule-destabilizing agent. Microtubule-stabilizing agents may be natural or synthetic.

Microtubule-stabilizing agents include but are not limited to: the taxanes including paclitaxel, docetaxel, 10-Deacetylbaccatin III, SB-T-1213, SB-T-1214, IDN5109, cabazitaxel, TX-67, BMS-275183, milataxel, and GRN 1005 (ANG1005); epothilones including epothilone A, epothilone B, epothilone C, epothilone D, epothilone E, epothilone F, fludelone, iaxbepilone, sagopilone, (E)-9, 10 dehydro-12,13-deoxy-EpoB, (E)-9, 10 dehydro-12,13-deoxy-EpoF and 26-F3-12,13-deoxyepothilone B; (+)-discodermolide; dictyostatin; eleutherobin; sarcodyctin A; sarcodyctin B; sarcodyctin C; sarcodyctin D; SKBIII.294; SKBIII.296; laulimalide and isolaulimalide; peloruside A and peloruside B; cyclostreptin; taccalonolide A; taccalonolide B; taccalonolide E; taccalonolide N; taccalonolide AF; taccalonolide AJ; zampanolide; dactylolide; ceratamine A and ceratamine B; dicumarol; jatrophane A; jatrophane B; jatrophane C; tubercidin; xanthophylls (e.g., lutein); the NAP peptide (also known as davunetide, which is a short peptide fragment NAPVSIPQ derived from the activity-dependent neuroprotective protein (ADNP); MT-stabilizing GS-164, estradiol derivative and SHPP-33; and a series of synthetic mono- and di-heterocyclic compounds with MT-stabilizing properties, including certain triazolopyrimidines, typified by cevipabulin (also known as TTI-237), as well as some structurally related phenylpyrimidines, pyridopyridazines, pyridotriazines and pyridazines; and pharmaceutically acceptable salts, acids and derivatives of any of the above.

Microtubule destabilizing agents include but are not limited to: vinca site binders such as the vinca alkaloids including vinblastine, vincristine, vinorelbine, vindesine, and vinflunine; cryptophycin 1; cryptophycin 24; cryptophycin 52; cryptophycin 55; dolastatin 10; dolastatin 15; eribulin; spongistatin; rhizoxin and tasidotin; colchicine-site binders including colchicine and its analogs; podophyllotoxin; combretastatins; CI-980; 2-methoxyestradiol; phenylahistins (diketopiperazine); steganacins, and curacins; hemiasterlin A and hemiasterlin B; estramustine; noscapine; herbicides such as carbendazim; psychoactive drugs such as phenytoin; and food components such as sulforaphane found in cruciferous vegetables; and pharmaceutically acceptable salts, acids and derivatives of any of the above.

In certain embodiments, the chemotherapeutic agent is mertansine (DM1) or DM4, or an analog, derivative, prodrug, or pharmaceutically acceptable salt thereof. DM1 or DM4 inhibits the assembly of microtubules by binding to tubulin.

In yet additional embodiments, the chemotherapeutic drug is monomethyl auristatin E [MMAE] or other dolastatins.

In certain embodiments, the chemotherapeutic agent is mertansine (DM1) or maytansine. Further examples of chemotherapeutic agents include: docetaxel; 5-FU (fluorouracil, 5-fluorouracil, CAS No. 51-21-8); gemcitabine; PD-0325901 (CAS No. 391210-10-9); cisplatin (cis-diamine, dichloroplatinum(II), CAS No. 15663-27-1); carboplatin (CAS No. 41575-94-4); trastuzumab; temozolomide (4-methyl-5-oxo-2,3,4,6,8-pentazabicyclo [4.3 0.0] nona-2,7,9-triene-9-carbox-amide, CAS No. 85622-93-1); doxorubicin; Akti-1/2; HPPD; and rapamycin.

More examples of chemotherapeutic agents include: oxaliplatin; bortezomib; chlorambucil; AG1478; AG1571 (SU 5271; Sugen); canfosfamide; thiotepa; cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan and irinotecan and SN-38); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); pancratistatin; spongistatin; nitrogen mustards such as chlorambucil; chlornaphazine; chlorophosphamide; estramustine; ifosfamide; mechlorethamine; mechlorethamine oxide hydrochloride; melphalan; novembichin; phenesterine; prednimustine; trofosfamide; uracil mustard; nitrosoureas such as carmustine, chlorozotocin, fotemustine, lomustine, nimustine, and ranimnustine; antibiotics such as the enediyne antibiotics (e.g., calicheamicin, calicheamicin gammall, and calicheamicin omegall); dynemicin; dynemicin A; esperamicin; neocarzinostatin chromophore and related chromoprotein enediyne antibiotic chromophores; aclacinomysins; actinomycin; authramycin; azaserine; bleomycins; cactinomycin; carabicin; carminomycin; carzinophilin; chromomycinis; dactinomycin; daunorubicin; detorubicin; 6-diazo-5-oxo-L-norleucine; morpholino-doxorubicin; cyanomorpholino-doxorubicin; 2-pyrrolino-doxorubicin and deoxydoxorubicin; epirubicin; esorubicin; idarubicin; marcellomycin; mitomycins such as mitomycin C; mycophenolic acid; nogalamycin; olivomycins; peplomycin; porfiromycin; puromycin; quelamycin; rodorubicin; streptonigrin; streptozocin; tubercidin; ubenimex; zinostatin; zorubicin; anti-metabolites such as methotrexate and 5-fluorouracil (5-FU); folic acid analogs such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; polysaccharide complex; razoxane; rhizoxin; sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside (Ara-C); cyclophosphamide; thiotepa; 6-thioguanine; mercaptopurine; methotrexate; etoposide (VP-16); ifosfamide; mitoxantrone; novantrone; teniposide; edatrexate; daunomycin; aminopterin; capecitabine; CPT-11; topoisomerase inhibitor RFS 2000; difluoromethylornithine (DMFO); rocaglamide or MZ735 or other inhibitors of eIF4A; and retinoids such as retinoic acid;

In some embodiments, the chemotherapeutic agent is an analog, derivative, prodrug, or pharmaceutically acceptable salt thereof of any of the above-listed agents.

In yet additional embodiments, the chemotherapeutic drug is SN-38, the active metabolite of the chemotherapy drug irinotecan.

Further examples of chemotherapeutic agents include peptides which are cytotoxic to cancer cells.

Proteins on the inner membrane of mitochondria that are essential for apoptosis can also serve as tumor therapeutic targets. The Bcl-2 family regulates the release of apoptotic-inducing factors. These factors can be divided into two groups: anti-apoptosis proteins such as bcl-2, bcl-XL and mc1-1; and pro-apoptosis proteins such as Bax, Bak, Bad, Bim and Bid. Peptides that mimic these proteins may bind to tumor cells and induce apoptosis, therefore achieving therapeutic effects in malignant tumor cells.

Peptides derived from the mitochondrial membrane-binding motif of Bax can cause cell apoptosis when conjugated with a cell-penetrating peptide. BH-3 mimetics navitoclax/ABT-737 and GX15-070 (obatoclax) have been developed. Navitoclax has shown cytotoxicity against selected hematological malignancies. Furthermore, the KLA peptide (KLAKLAK)2 (SEQ ID NO: 56) can cause damage to mitochondrial membranes and induce cell apoptosis. Additionally, after binding to tumor-homing peptides, the therapeutic effect of mitochondriotoxic peptides can be greatly improved. For example, the KLA peptide has been fused to TTPs such as RGD, PTP, and TCTP. These conjugates can be made with the non-hydrolyzable, non-cleavable, stable linker disclosed herein.

Other peptides for use as a chemotherapeutic agent include but are not limited to cecropin A and B, pleurocidin, magainin 2, and β-defensin. See Boohaker et al. 2012.

### Other Therapeutic Agents

As discussed above, targeting moieties for cell surface markers implicated in other diseases can be used such as a peptide (DEMEFTEAESNMN) (SEQ ID NO: 51) that targets the G protein-coupled receptor kinase implicated in heart disease and brain diseases such as Alzheimer's disease. See Asai et al. 2014. Thus, therapeutic agents which treat other diseases such as heart disease and Alzheimer's disease can also be used in the disclosed compositions.

Non-Hydrolyzable, Non-Cleavable, Stable linkers

The conjugate composition may contain one or more linkers attaching the targeting moiety and the chemotherapeutic drug. The linker may be attached to the targeting moiety and the chemotherapeutic drug by functional groups independently selected from an ester bond, disulfide, amide, acylhydrazone, ether, carbamate, carbonate, and urea. Alternatively, the linker can be attached to either the targeting moiety or the chemotherapeutic drug by a non-cleavable group such as provided by the conjugation between a thiol and a maleimide, an azide and an alkyne. In certain embodiments, the linker is independently selected from the group consisting alkyl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl, wherein each of the carboxyl, carbamoyl, ether, alkoxy, aryloxy, amino, amide, carbamate, alkyl, alkenyl, alkynyl, aryl, arylalkyl, cycloalkyl, heteroaryl, or heterocyclyl is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl.

In some embodiments the alkyl chain of the linker may optionally be interrupted by one or more atoms or groups selected from —O—, —C(═O)—, —NR, —O—C(═O)—NR—, —S—, —S—S—. The linker may be selected from dicarboxylate derivatives of succinic acid, glutaric acid or diglycolic acid.

In some embodiments, the targeting moiety contains an amino acid capable of making an amide bond. In some embodiments, the linker is bound to the targeting moiety via an amide bond, i.e., —NH—CO—, or —CO—NH— (the hydrogen on the nitrogen may be substituted). In some embodiments, the linker is not bound to the SSTR-targeting moiety via an amide bond. In some embodiments, the linker includes an amide bond, i.e., —NH—CO—, or —CO—NH— (the hydrogen on the nitrogen may be substituted).

In certain embodiments, the linker is chosen from the group consisting of GMBS and PEG.

In certain embodiments, the linker is a chemical linker, including, but not limited to the compounds structures shown in (A), (B), and (C) wherein n is an integer of 1 or more.

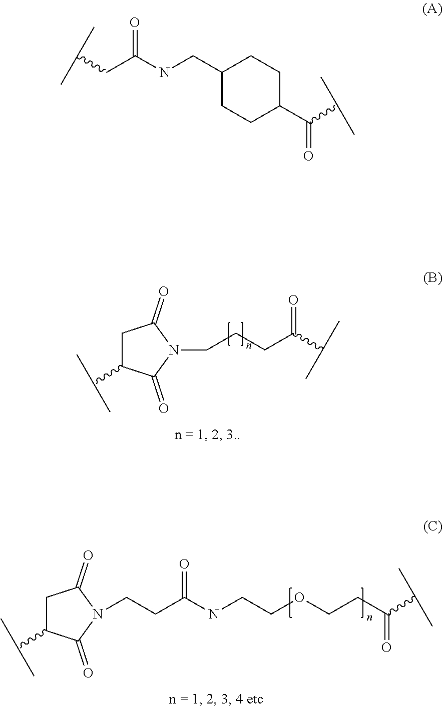

In some embodiments, the linkers are non-cleavable and non-hydrolyzable, meaning that the composition does not release the chemotherapeutic drug in vivo. Importantly, the chemotherapeutic drug is neither released extracellularly such as in the circulation nor released inside a cell.

In some embodiments, the composition comprising the targeting moiety (e.g., lanreotide), the chemotherapeutic agent or drug (e.g., DM1) and the linker is delivered to the cell. The chemotherapeutic agent or drug, e.g., microtubule-targeting agent (MTA) or tubulin-targeting agent, binds to the tubulin in the cells and kills the cells with the conjugate still intact. Outside the cell the chemotherapeutic agent is not released from the conjugate and thus can cause no harm to other cells.

The fact that the chemotherapeutic or therapeutic agent is not released prior to reaching the targeting cell also means that the agent is more effective and less might be needed to treat the cancer or other disease. The current compositions with the non-hydrolyzable, non-cleavable, stable linker allow more toxic and potent chemotherapeutic agents to be used because the potent chemotherapeutic agent is delivered to the target cell only and does not enter the bloodstream or delivered to healthy tissue.

### Therapeutic Indications and Uses

The compositions and therapeutic methods described herein can be used for the treatment of lymphoma.

In certain embodiments, lymphoma includes non-Hodgkin lymphoma.

In some embodiments, the cancer is neuroendocrine prostate cancer.

In some embodiments, the neuroendocrine prostate cancer is not clinically characterized as a neuroendocrine prostate cancer, but rather as prostate cancer that is resistant to anti-hormonal agents. In this instance, the target is the somatostatin receptor that is expressed in the prostate cancer cells of a cancer state not yet clinically designated neuroendocrine prostate cancer.

Further non-limiting examples of types of cancers/tumors for treatment using the conjugate compositions disclosed herein include small lymphocytic lymphoma, lymphoplasmacytic B-cell lymphoma, Waldenström macroglobulinemia, splenic marginal zone lymphoma, plasmacytoma, extranodal marginal zone B cell lymphoma, MALT lymphoma, nodal marginal zone B cell lymphoma (NMZL), follicular lymphoma, mantle cell lymphoma, diffuse large B cell lymphoma (DLBCL), mediastinal (thymic) large B cell lymphoma, intravascular large B cell lymphoma, primary effusion lymphoma, Burkitt lymphoma, chronic lymphocytic lymphoma (CLL), adult T cell lymphoma, nasal type extranodal NK/T cell lymphoma, enteropathy-type T cell lymphoma, hepatosplenic T cell lymphoma, blastic NK cell lymphoma, mycosis fungoides, Sezary syndrome, primary cutaneous CD30-positive T cell lymphoproliferative disorders, primary cutaneous anaplastic large cell lymphoma, lymphomatoid papulosis, angioimmunoblastic T cell lymphoma, unspecified peripheral T cell lymphoma, and anaplastic large cell lymphoma. Additionally, the present disclosure includes refractory or recurrent malignancies whose growth may be inhibited using the conjugates described herein.

The compositions and therapeutic methods described herein can be used for the treatment of additional cancers including but not limited to sarcoma, neuroblastoma, glioblastoma, melanoma, lung carcinoma, non-small cell lung cancer, glioma, head and neck cancer, prostate cancer, colorectal cancer, liver cancer, ovarian cancer, pancreatic cancer, squamous cell cancer, mesothelioma, breast cancer, brain cancer, cervical cancer, stomach cancer, and leukemia as well as neuroendocrine tumors or carcinoid tumors.

A diverse group of cancers can be treated using the described conjugate compositions because standard of care imaging techniques can be leveraged to identify those cancers with the required receptor making this therapy tissue agnostic. Furthermore, cancers that may not express the receptor under normal circumstances, but in which the expression of the receptor can be induced, even if transiently, can be include amongst those cancers for which the compositions and therapeutic methods described herein can be used for the treatment. Induction of the expression of receptors can be achieved, even if transiently, with the use of epigenetic agents or drugs including many with regulatory approvals. Transient induction can be sufficient since it allows for the delivery to the inside of the cell the compositions and therapeutic methods described herein.

The compositions and therapeutic methods described herein can be used for the treatment of additional diseases including but not limited to heart disease and brain diseases such as Alzheimer's disease.

### Combination Therapy

The present conjugate can be given subsequent to, preceding, or contemporaneously with other therapies including cancer therapies. For example, the subject may previously or concurrently be treated by chemotherapy, radiation therapy, surgery, immunotherapy, anti-angiogenic agents, anti-viral agents, and hormonal agents. Additionally, the subject may be treated concurrently with a long acting or slow release somatostatin receptor targeting moieties including but not limited to octreotide or lanreotide.

In some embodiments, the method further comprises treating the patient with an agent which induces or increases the expression of a receptor on the target cells. In some embodiments, the receptor is the somatostatin receptor. In some embodiments, the receptor is SSTR2. The agent which increases the levels of the receptor on the surface of the cancer cell, even if transiently, usually belongs to a class of drug commonly referred to as epigenetic modifiers. In some embodiments, the agent is an epigenetic modifier or a combination of epigenetic modifiers. In some embodiments, the agent is a DNA methylation inhibitor including but not limited to 5-aza-2′deoxycytidine or decitabine. In some embodiments, the agent is a histone deacetylase inhibitor including but not limited to trichostatin A, romidepsin also known as Istodax, belinostat, entinostat, panobinostat and vorinostat, as well as other similar agents that can increase the receptor levels by increasing histone acetylation. In some embodiments the agent is a histone methyltransferase inhibitor, including but not limited to tazemetostat or pinometostat. In some embodiments the agent is an IDH1/2 inhibitor including but not limited to ivosidenib and enasidenib. In some embodiments the agent is a histone acetyltransferase inhibitor. In some embodiments, the agent is a histone demethylase inhibitor including but not limited to GSK2879552 or tranylcypromine. In some embodiments, the agent is a bromodomain and extraterminal domain (BET) protein inhibitor including but not limited to molibresib. Other epigenetic modifiers are in development and can also be envisioned to be useful in future embodiments, as epigenetic modifiers change gene expression.

### Pharmaceutical Compositions and Administration

The compositions disclosed herein can be formulated according to known methods for preparing pharmaceutically acceptable compositions. The phrase “pharmaceutically acceptable” refers to molecular entities and compositions that are physiologically tolerable and do not typically produce an allergic or similar untoward reaction, such as gastric upset, dizziness and the like, when administered to a human, and approved by a regulatory agency of the Federal or a state government or listed in the U.S. Pharmacopeia or other generally recognized pharmacopeia for use in animals, and more particularly in humans. Formulations are described in detail in a number of sources that are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use.

Methods of administration include oral; mucosal, such as nasal, sublingual, vaginal, buccal, or rectal; parenteral, such as subcutaneous, intravenous, bolus injection, intramuscular, or intra-arterial; or transdermal administration to a subject.

A preferred method of administration is injection or a depot formulation as long-acting/depot formulations of lanreotide and octreotide are approved by the FDA. This method of administration allows for the conjugate composition to be effectively administered continuously, a property that is desirable for many chemotherapies and especially so for those targeting the microtubules. Such continuous administration can be especially valuable in combination regimens where drugs that damage DNA are used or where radiotherapy is administered such as is the case with peptide receptor radionuclide therapy (PRRT), for example lutetium Lu177 dotatate (Lutathera®), or other forms of PRRT.

Selection of a therapeutically effective amount or dose will be determined by the skilled artisan considering several factors which will be known to one of ordinary skill in the art. Such factors include the particular form of the inhibitor, and its pharmacokinetic parameters such as bioavailability, metabolism, and half-life, which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

With regards to dosing two principal factors are to be considered. The first is the quantity of the conjugate compositions of the invention to be administered and the second is the timing or frequency of administration. The maximum dose that will be tolerated will be established in conventional phase I trials performed for the purposes of assessing that dose that is tolerated without many adverse effects. Given the expectation that the conjugate composition will not be hydrolyzed as it is stable, and that its delivery to cells is determined by the targeting moiety, the amount that will be tolerated in many cases may be the amount of the targeting moiety that can be administered. In the case of lanreotide, for example, with the total molecular weight of the conjugate composition of the invention about 2.5 times that of the targeting moiety—lanreotide—this could be 120 mg multiplied by 2.5 or 300 mg every four weeks either as a single injection in a depot formulation or in divided doses with the dose administered on a regular basis, for example every week or every other week or every third week or every fourth week to be about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% of the total 300 mg dose.

Similarly in the case of octreotide whose usually accepted maximal dose when administered as a long-acting release formulation is 30 mg or in some cases 40 mg or 50 mg or 60 mg, the total dose could be 60 mg multiplied by 2.5 or 150 mg every four weeks either as a single injection in a depot formulation or in divided doses with the dose administered on a regular basis, for example every week or every other week or every third week or every fourth week to be about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% of the total 150 mg dose.

The composition of the present invention can be administered at any time that is appropriate. For example, the administration can be conducted before or during traditional therapy of a subject having cancer (e.g., lymphoma), and continued after the cancer (e.g., lymphoma) becomes clinically undetectable. The administration also can be continued in a subject showing signs of recurrence.

### EXAMPLES

The present invention may be better understood by reference to the following non-limiting examples, which are presented in order to more fully illustrate the preferred embodiments of the invention. They should in no way be construed to limit the broad scope of the invention.

### Example 1-Choice of Target and Chemotherapeutic Agent

Malignant lymphomas emerged as especially attractive since: (1) high levels of SSTR expression are common amongst lymphomas but has not been previously emphasized therapeutically and thus represents a highly novel target for precision therapy (Lugtenburg et al. 2001; Witzig et al. 1995); (2) lymphomas are generally chemoresponsive and microtubule-targeting agents (MTAs) are components of the majority of regimens underscoring microtubules (MTs) as a valid target (Rai et al. 2015; Ruscica et al. 2013); and (3) neurotoxicity is encountered as a frequent complication of standard therapies, making administration of a potent MT-targeting agent in a precise manner that should avoid this complication very attractive.

Mertansine, a thiol-containing maytansinoid, can attach to another moiety through reaction of the thiol group with a linker to create a drug conjugate. An example of the somatostatin analogs is lanreotide where initial experiments confirmed its effectiveness. Specifically, this strategy recognizes that: (1) targeting the SSTR is a validated clinical strategy that has proven very tolerable; (2) delivery of cytotoxic payloads to human tumors is also a validated strategy; (3) the mitotic spindle is likely a target in very rapidly dividing lymphomas with interphase microtubules (MTs) as the primary target of MTAs in the majority of lymphomas; and (4) continuous delivery might achieve greater efficacy.

Choice of the target: Five genes encode the somatostatin receptors (SSTRs)—SSTR1, 2, 3, 4 and 5. All are G protein coupled, seven-trans-membrane domain receptors. Somatostatin, considered an inhibitory hormone but still poorly understood, binds the SSTRs with roughly equal affinity and inhibits the secretion of hormones including gastrin, insulin, and secretin and vasoactive intestinal peptide (Rai et al. 2015; Ruscica et al. 2013). Synthetic analogues, particularly octreotide and lanreotide are more potent inhibitors of hormone secretion, bind with higher affinity to SSTR2 and are used clinically to prevent the systemic effects of hormone producing neuroendocrine tumors (NETs). In addition, these analogues slow the growth of NETs, and are used to control disease in patients without hormone excess (Caplin et al. 2014; Strosberg et al. 2017). Long-acting/depot formulations of lanreotide and octreotide are approved by the FDA and in the case of lanreotide were shown in a randomized trial that led to its approval in the NET indication, to increase progression-free survival in NETs. Similar data is available for octreotide albeit of less robust quality. Radiolabeled formulations, including ⁹⁰Y-octreotide and ¹⁷⁷Luoctreotate are also employed in treatment of NETs.

These data suggested that the SSTR was a viable target that can be further exploited and whose interdiction is therapeutically tolerable. An initial question was to determine which of the five SSTRs are appropriate for targeting lymphoma. The agonists lanreotide and octreotide preferentially bind SSTR2. A survey of publicly available databases—the Cancer Genome Atlas (TCGA), and the Cancer Cell Line Encyclopedia (CCLE)—demonstrated high levels of SSTR2 expression in malignant lymphomas.

Together with the data that with the exception of brain tissue, normal tissue expression of SSTR2 is low, these unbiased analyses were consistent with clinical observations showing a good therapeutic window for agonists targeting SSTR2 and support the expected safety of drug conjugates targeting SSTR2. Normal tissue toxicity from drug conjugates targeted by somatostatin analogues was not expected.

The cytoskeleton of eukaryotic cells participates in various cellular functions. Microtubules (MTs) are an integral part of the cytoskeleton. Among anti-cancer agents, drugs targeting tubulin/MTs are amongst the most effective agents. Drugs targeting tubulin/MTs may be natural or synthetic, with diverse chemical structures. The knowledge that “traditional cytotoxic agents” have benefited, and even cured many patients with malignant lymphomas supports continued interest in these compounds.

To explain the activity of MTAs in human tumors that divide much more slowly than pre-clinical models, interfering with microtubule trafficking in interphase cells has been proposed (Komlodi-Pasztor et al. 2011; Komlodi-Pasztor et al. 2012). The trafficking of essential proteins on MTs has been evaluated. Data showed that trafficking in general is important, including trafficking of DNA repair proteins. It has previously been demonstrated that by hampering the trafficking of essential DNA repair proteins, MTAs synergize with DNA damaging agents (DDAs), and with radiation therapy, augmenting their toxicity (Poruchynsky et al. 2015). Given that both MTAs and DDAs are integral components of nearly all existing curative lymphoma regimens, a precise agent targeting MTs is an attractive agent for the therapy of malignant lymphomas.

Note here that the same can be said for the majority of solid tumors making the therapies described herein relevant across the spectrum of solid tumors as well as hematological malignancies. Note here also that a therapy such as this could be beneficial in cases where expression of the somatostatin receptor is not generally recognized as is the case in prostate cancer that has progressed after treatment with anti-hormonal agents, even before it is clinically recognized as a neuroendocrine prostate cancer. Note also that a therapy such as this could be beneficial in cases where the expression of the somatostatin receptor can be induced, even if transiently, by agents that target the epigenetic landscape of a cancer cell. Even transient induction would allow for the intracellular delivery of the potent chemotherapeutic.

### Example 2-Synthesis of Conjugate Composition DM1-GMBS-Lanreotide (P182-1-DM1)

Conjugate composition comprising DM1 and lanreotide and the non-hydrolyzable, non-cleavable, stable linker GMBS was synthesized as shown in Scheme 1. Briefly, the primary amine of lanreotide was protected with Boc20. The reaction of the second amine group of the protected lanreotide with the NHS ester of GMBS gave the intermediate GMBS-lanreotide. The reaction of the maleimide of GMBS-lanreotide with the sulfhydryl group (—SH) of DM1 gave a protected conjugate. De-protection of the Boc with TFA provided the final product. All compounds were fully characterized and purified to >95% as determined by NMR and LC-MS analyses. The purity of the compound for animal studies is >98%. Aqueous solutions of at least one millimolar can be achieved and preliminary studies indicate are stable at 4° C. for months.

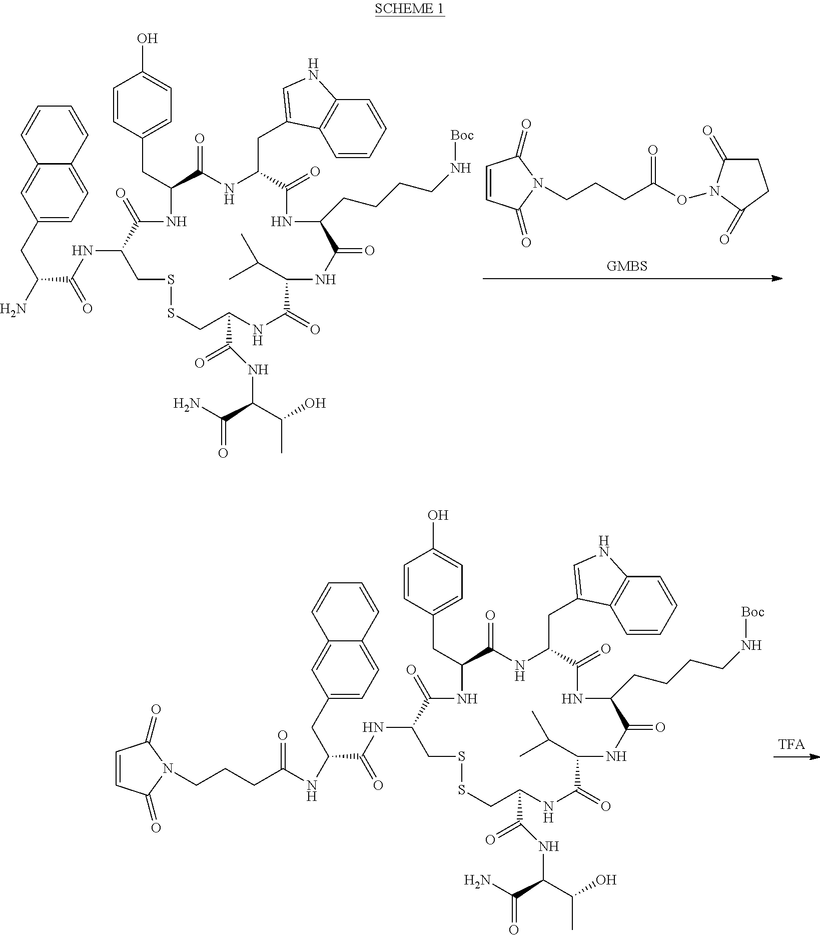

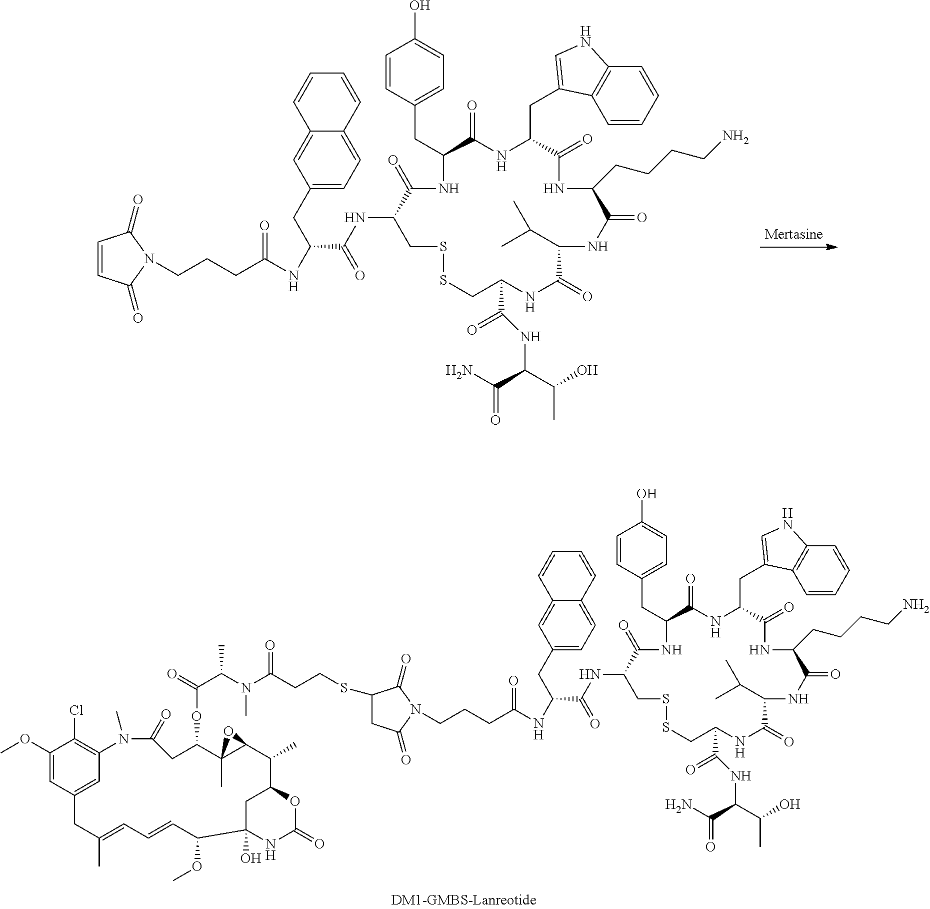

### Example 3-Synthesis of Conjugate Composition DM1-PEG-Lanreotide (P182-2-DM1)

Conjugate composition comprising DM1 and lanreotide and the non-hydrolyzable, non-cleavable, stable linker PEG was synthesized as shown in Scheme 2. Briefly, the primary amine of lanreotide was protected with Boc20. The reaction of the second amine group of the protected lanreotide with the NHS ester of PEG gave the intermediate PEG-lanreotide. The reaction of the maleimide of PEG-lanreotide with the sulfhydryl group (—SH) of DM1 gave a protected conjugate. De-protection of the Boc with TFA provided the final product. All compounds were fully characterized and purified to >95% as determined by NMR and LC-MS analyses. The purity of the compound for animal studies is >98%. Aqueous solutions of at least one millimolar can be achieved and preliminary studies indicate are stable at 4° C. for months.

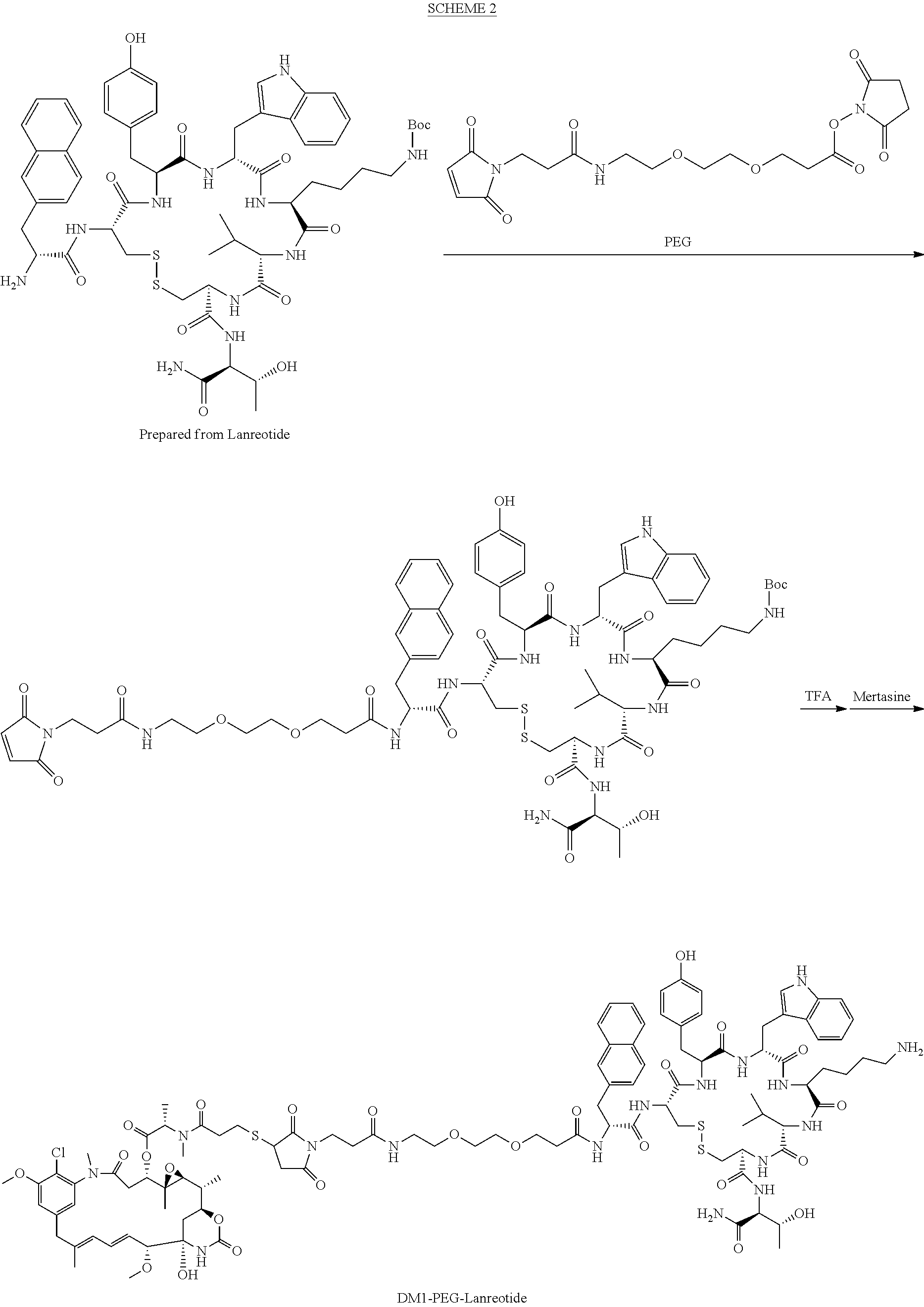

### Example 4—Conjugate Compositions were Active and Effective in Malignant Lymphoma Cells

### Materials and Methods

Malignant lymphoma cell lines Pffifer and Karpas were used as previously described (Zhuang et al. 2007; Huang et al. 2010; Burotto et al. 2015). The compositions described in Example 2 and 3 were added to the media in concentrations ranging from 10 μM to 37 nM. Cells were then incubated in media with the varying concentrations of drug or without drug at 37° C. under 5% CO₂ for 72-96 hours after which time cellular viability was determined using CellTiter-Glo Luminescent Cell Viability assay.

SSTR2 expression was measured in the cells using antibodies. Cells were lysed using RIPA buffer, and protein loaded onto NuPAGE 4-12% Bis-Tris protein gels, electrophoresed and transferred to nitrocellulose membrane. Membranes were incubated overnight at 4° C. with primary antibodies: GAPDH (1:10000, Abcam # ab8245); SSTR2 (A-8) (1:200, Santa Cruz Biotechnology # sc-365502), washed with Tris Buffered Saline and then incubated with Li-Cor secondary antibody conjugate IRDye® 680RD Goat anti-Mouse.

### Results

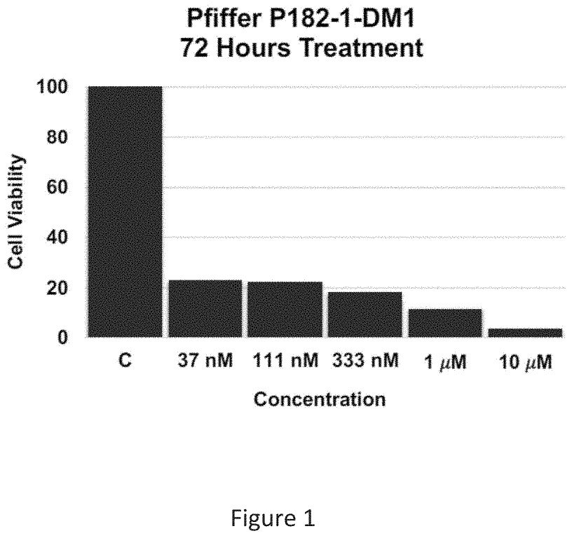

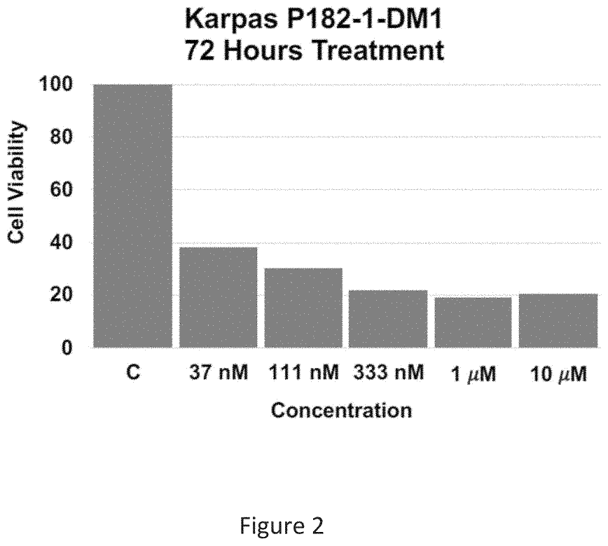

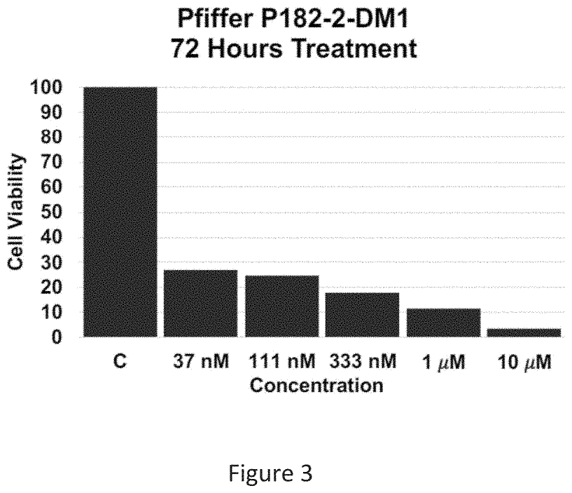

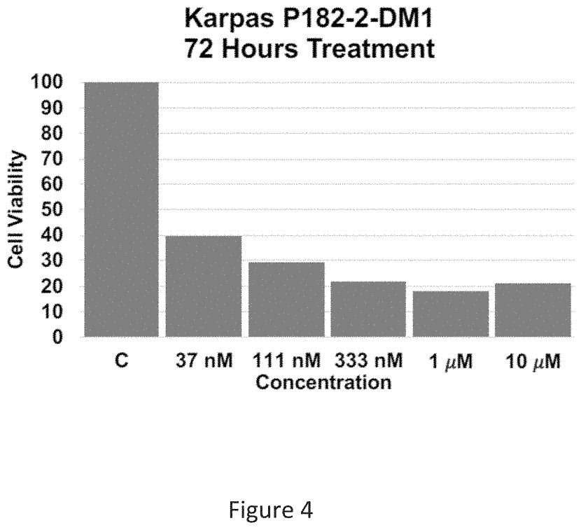

As shown in FIGS. 1-4, both conjugates were active in, i.e., killed, the cells in a dose dependent manner at only 72 hours.

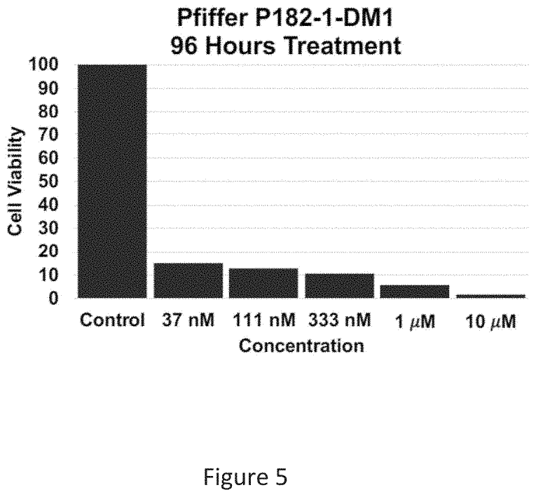

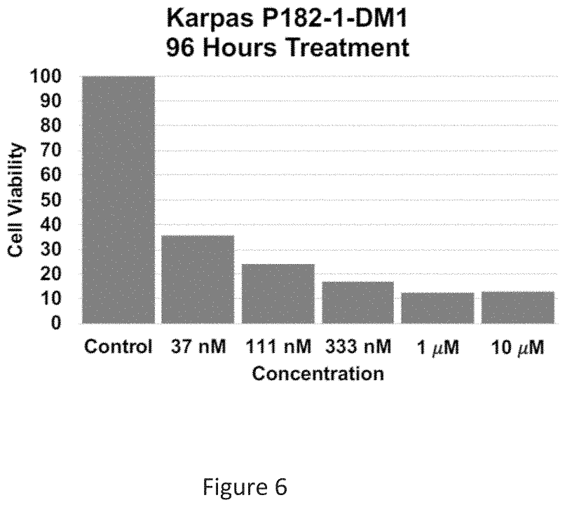

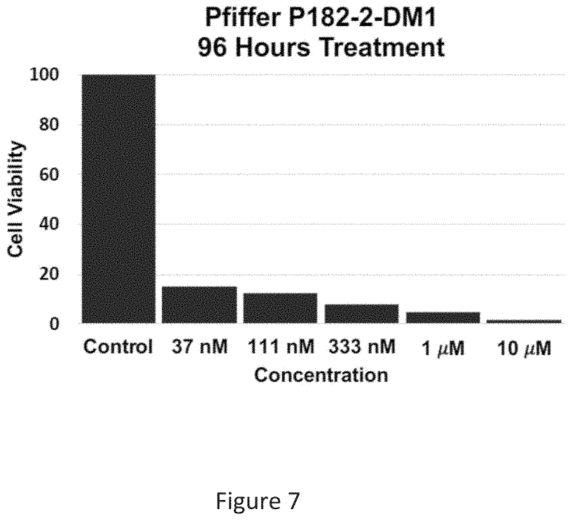

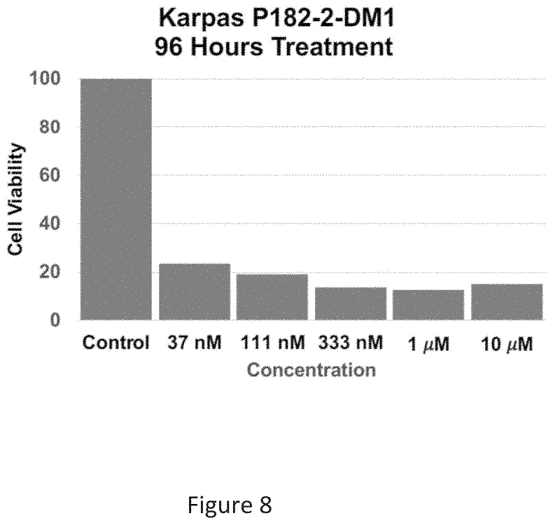

The results are more striking at 96 hours with both conjugates killing almost all of the cells in Pffifer cell line at 10 μM. See FIGS. 5-8.

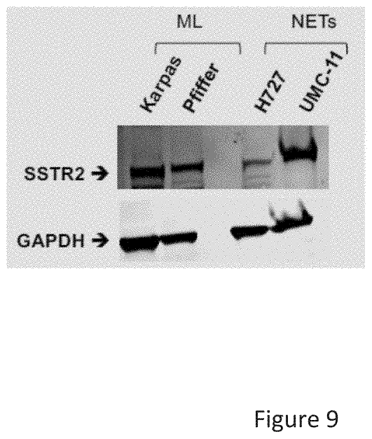

The results showed that the intact compositions comprised of the targeting moiety, the chemotherapeutic agent and the non-hydrolyzable, non-cleavable, stable linker was effective in killing cells with the target, in this case, SSTR2. As shown in FIG. 9, SSTR2 expression in malignant lymphoma cells (ML) was comparable to NETs for which lanreotide is used clinically to target.

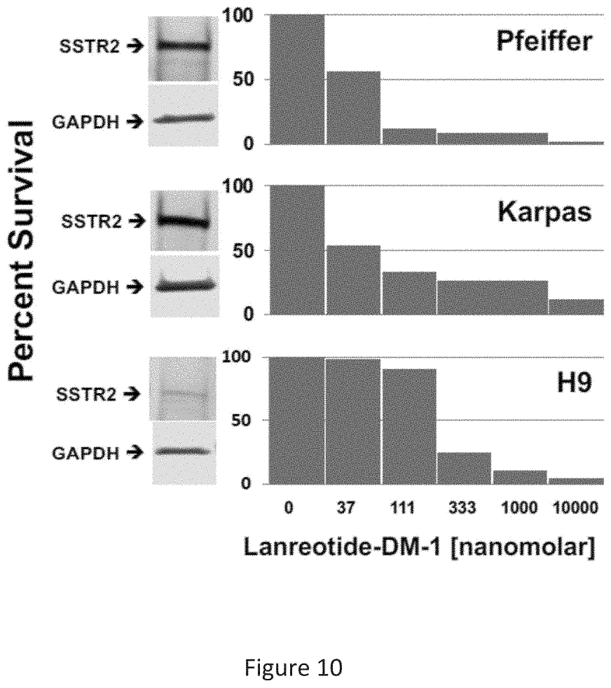

As shown in FIG. 10, conjugates comprising lanreotide and DM1 were active, i.e., killed, the cells in Pfiffer and Karpas cell lines, and to a lesser extent against H9 cells that express lower levels of SSTR2.

### Example 5-Induction of the Somatostatin Receptor Provides a Novel Approach to Deliver Greater Quantities of Conjugate Compositions

### Materials and Methods

Three neuroendocrine cell lines, NEC1 (NCI—H82), NEC2 (Kelly), and NEC3 (H727), were incubated in media containing 1 or 3 nanomolar romidepsin (EA1) or 1 micromolar entinostat (EA2) or without drug at 37° C. under 5% CO₂ for 72-96 hours after which time cellular RNA was harvested and expression of the SSTR determined.

Somatostatin receptor expression was measured by quantitative PCR using primers for SSTR2.

### Results

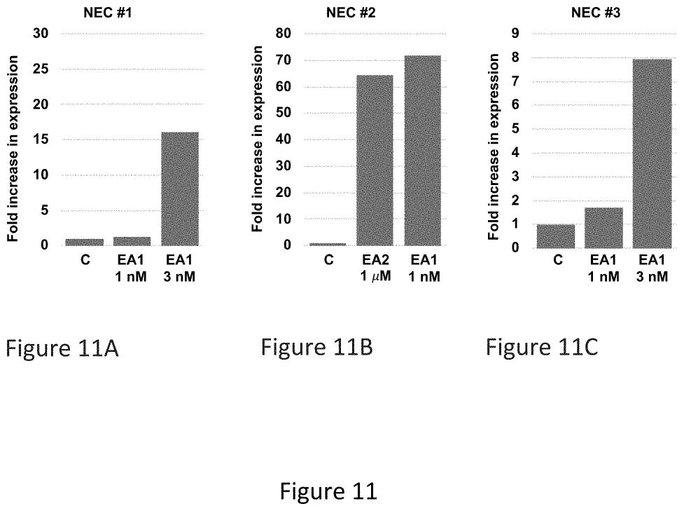

Induction of the somatostatin receptor in three neuroendocrine cancer cell lines [NEC1 (NCI—H82), NEC2 (Kelly), NEC3 (H727)] with two epigenetic agents [EA1 (romidepsin), EA2 (entinostat)] within 72 hours at concentrations that are not cytotoxic or only minimally cytotoxic was observed (FIG. 11).

Induction such as that observed here, even if transient, provides a novel approach for the use of the peptide drug conjugates as it allows for delivery of the cytotoxic DM1 to cells that under normal circumstances do not express the somatostatin receptor and would not be otherwise sensitive. This then expands the indications for the therapies described herein, such that they can be used under circumstances or in the treatment of cancer or cancer cells where expression of the somatostatin receptor is not present or only present at low levels and in which the expression of the receptor can be induced, even if transiently, with epigenetic agents.

### Example 6—Conjugate Compositions were Active and Effective in Neuroendocrine Prostate Cancer

### Materials and Methods

A neuroendocrine prostate cancer (NEPC) cell line was used and NEPC organoids were generated.

NEPC organoids were treated for three days with DM1 alone, a conjugate of lanreotide, DM1 and cleavable, hydrolysable linker, MCC (maleimidomethyl cyclohexane-1-carboxylate), and the composition described in Example 3, 182-2-DM1 at concentrations of 1 uM, 0.33 uM, 0.11 uM, 0.037 uM, 0.012 uM, 0.0041 uM, 0.00137 uM and 0.00046 uM. CellTiter-Glo Luminescent Cell Viability assay measured viable cells. The percentage of viable cells after drug treatment compared to vehicle control (DMSO) was used to generate a dose—response curve.

Expression of SSTR2 was measured in the NEPC organoid, brain lysate and the Pfiffer cells used in Example 4.

### Results

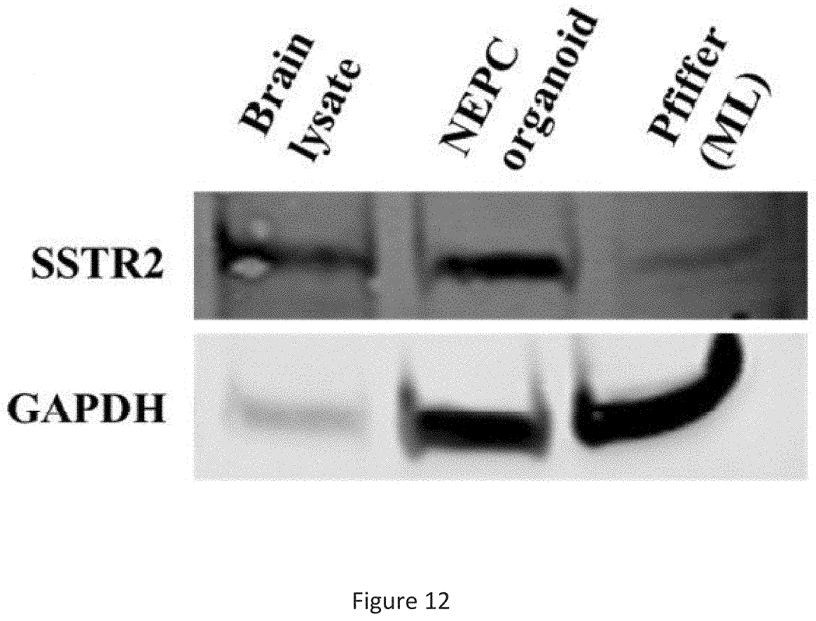

As shown in FIG. 12, an anti-SSTR2 antibody demonstrated robust expression of SSTR2 in a neuroendocrine prostate cancer (NEPC).

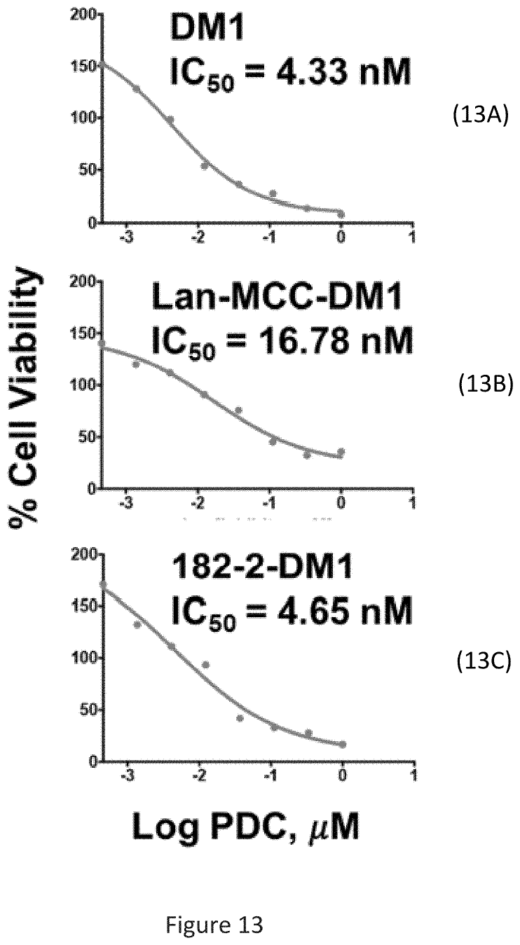

As shown in FIG. 13, the conjugate composition 182-2-DM1 was effective and active in the NEPC organoids and more effective than a conjugate where a cleavable, hydrolysable linker was used. Moreover, the IC₅₀ value for 182-2-DM1 was similar to DM1 meaning that the activity of the cytotoxic payload in the conjugate was retained in its entirety.

### REFERENCES

Asai et al. Peptide Substrates for G-protein-coupled receptor kinase 2. FEBS Letters 2014; 588: 2129-32
Boohaker et al. The Use of Therapeutic Peptides to Target and to Kill Cancer Cells. Curr Med Chem 2012; 19(22);3794-3804
Burotto et al. Phase II Clinical Trial of Ixabepilone in Metastatic Cervical Carcinoma. Oncologist. 2015; 20:725-6
Caplin et al. for CLARINET Investigators. Lanreotide in metastatic enteropancreatic neuroendocrine tumors. N Engl J Med. 2014; 371:224-33
Deng et al. The novel IKK2 inhibitor LY2409881 potently synergizes with histone deacetylase inhibitors in preclinical models of lymphoma through the downregulation of NF-kappaB. Clin Cancer Res. 2015; 21:134-145
Huang et al. A phase II clinical trial of ixabepilone (Ixempra; BMS-247550; NSC 710428), an epothilone B analog, in patients with metastatic renal cell carcinoma. Clin Cancer Res. 2010; 16:1634-41
Komlodi-Pasztor et al. Mitosis is not a key target of microtubule agents in patient tumors. Nat Rev Clin Oncol. 2011; 8:244-50
Komlodi-Pasztor et al. Inhibitors targeting mitosis: tales of how great drugs against a promising target were brought down by a flawed rationale. Clin Cancer Res. 2012; 18:51-63
Lugtenburg et al. Somatostatin receptor scintigraphy in the initial staging of low-grade non-Hodgkin's lymphomas. J Nucl Med. 2001; 42:222-9
Newell et al. Evaluation of rodent-only toxicology for early clinical trials with novel cancer therapeutics. British Journal of Cancer 1999; 81:760-768.
Poruchynsky et al. Microtubule-targeting moieties augment the toxicity of DNA-damaging agents by disrupting intracellular trafficking of DNA repair proteins. Proc Natl Acad Sci USA. 2015; 112:1571-6
Rai et al. Therapeutic uses of somatostatin and its analogues: Current view and potential applications. Pharmacol Ther. 2015; 152:98-110.
Ruscica et al. Somatostatin, somatostatin analogs and somatostatin receptor dynamics in the biology of cancer progression. Curr Mol Med. 2013; 13:555-71.
Strosberg et al. NETTER-1 Trial Investigators. Phase 3 Trial of ¹⁷⁷Lu-Dotatate for Midgut Neuroendocrine Tumors. N Engl J Med. 2017; 376:125-135.
Witzig et al. Evaluation of a somatostatin analog in the treatment of lymphoproliferative disorders: results of a phase II North Central Cancer Treatment Group trial. J Clin Oncol. 1995; 13:2012-5.
Xiao et al. Peptide-Based Treatment: A Promising Cancer Therapy. Journal of Immunology Research 2015; Article ID 761820
Zhao et al. Tumor-targeting Peptides: Ligands for Molecular Imaging and Therapy. Anti-Cancer Agents in Medicinal Chemistry 2018; 18:74-86.
Zhuang et al. Evidence for microtubule target engagement in tumors of patients receiving ixabepilone. Clin Cancer Res. 2007; 13:7480-6.

1.-3. (canceled)

4. A composition comprising a non-hydrolyzable non-cleavable, stable linker having the structure: 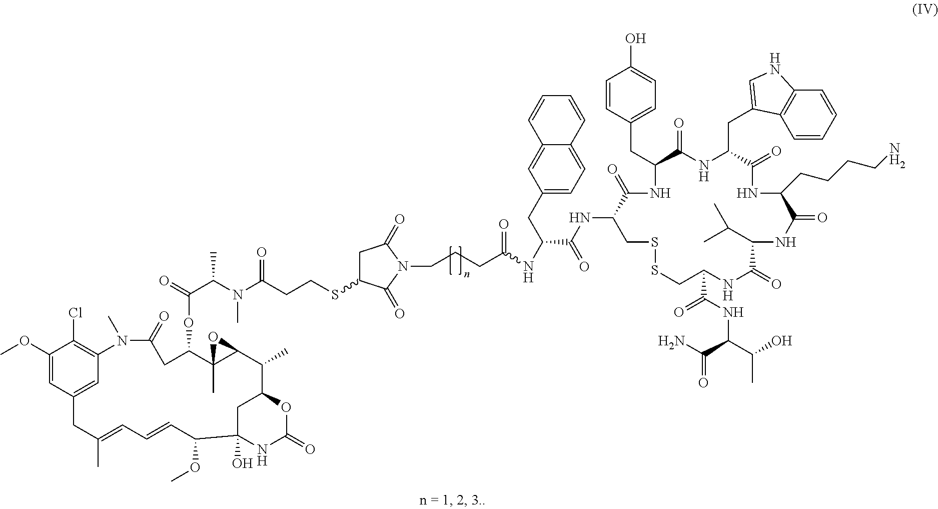
wherein n is an integer of 1 or more.

5. A composition comprising a non-hydrolyzable non-cleavable, stable linker having the structure: 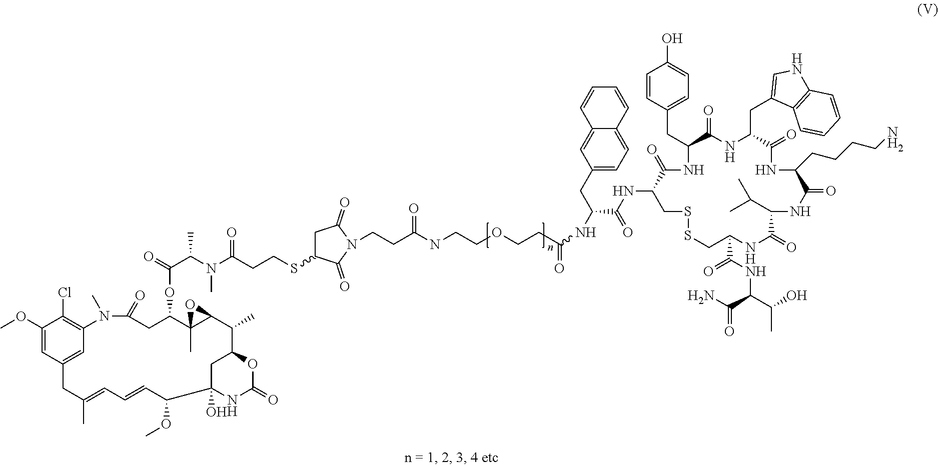
wherein n is an integer of 1 or more.

6. A composition comprising: a non-hydrolyzable non-cleavable, stable linker chosen from the group consisting of GMBS, PEG, and the structures (A), (B), and (C); targeting moiety; and a chemotherapeutic agent.

7. The composition of claim 31, wherein the SSTR-targeting moiety is chosen from the group consisting of SSTR1, SSTR2, SSTR3, SSTR4 and SSTR5.

8.-10. (canceled)

11. The composition of claim 31, wherein the SSTR-targeting moiety is a peptide.

12. The composition of claim 31 wherein the SSTR-targeting moiety is selected from the group consisting of lanreotide, octreotide, octreotate, pasireotide, vapreotide, seglitide, and derivatives thereof.

13. The composition of claim 6, wherein the chemotherapeutic agent targets microtubules.

14. The composition of claim 13, wherein the chemotherapeutic agent is a microtubule-destabilizing drug or a microtubule-stabilizing drug.

15. The composition of claim 14, wherein the chemotherapeutic agent is mertansine (DM1), DM4, maytansine, or an analog, derivative, prodrug, or pharmaceutically acceptable salt thereof.

16. A method of treating cancer in a subject in need thereof, comprising administering to the subject a therapeutically effective amount of the composition of claim 6.

17. The method of claim 16, further comprising administering to the subject a therapeutically effective amount of an agent which induces the expression of a receptor on the target cells.

18. The method of claim 17, wherein the receptor is a somatostatin receptor (SSTR).

19. The method of claim 17, wherein the agent which induces the expression of the receptor on the target cells is an epigenetic agent chosen from the group consisting of DNA methylation inhibitors, histone deacetylase inhibitors, histone methyltransferase inhibitors, IDH1/2 inhibitors, histone acetyltransferase inhibitors, histone demethylase inhibitors, bromodomain and extraterminal domain (BET) protein inhibitors, and combination thereof.

20. The method of claim 16, wherein the cancer is chosen from the group consisting of lymphoma, sarcoma, neuroblastoma, glioblastoma, melanoma, lung carcinoma, non-small cell lung cancer, glioma, head and neck cancer, prostate cancer, colorectal cancer, liver cancer, ovarian cancer, pancreatic cancer, squamous cell cancer, mesothelioma, breast cancer, brain cancer, cervical cancer, stomach cancer, and leukemia, neuroendocrine tumors and carcinoid tumors.

21. The method of claim 20, wherein the lymphoma is chosen from the group consisting of non-Hodgkin lymphoma (NHL), small lymphocytic lymphoma, lymphoplasmacytic B cell lymphoma, Waldenström macroglobulinemia, splenic marginal zone lymphoma, plasmacytoma, extranodal marginal zone B cell lymphoma, MALT lymphoma, nodal marginal zone B cell lymphoma (NMZL), follicular lymphoma, mantle cell lymphoma, diffuse large B cell lymphoma (DLBCL), mediastinal (thymic) large B cell lymphoma, intravascular large B cell lymphoma, primary effusion lymphoma, Burkitt lymphoma, chronic lymphocytic lymphoma, adult T cell lymphoma, nasal type extranodal NK/T cell lymphoma, enteropathy-type T cell lymphoma, hepatosplenic T cell lymphoma, blastic NK cell lymphoma, mycosis fungoides, Sezary syndrome, primary cutaneous CD30-positive T cell lymphoproliferative disorders, primary cutaneous anaplastic large cell lymphoma, lymphomatoid papulosis, angioimmunoblastic T cell lymphoma, unspecified peripheral T cell lymphoma, and anaplastic large cell lymphoma.

22. The method of claim 20, wherein the lymphoma is non-Hodgkin lymphoma.

23. The method of claim 16, wherein the cancer is neuroendocrine prostate cancer or a precursor state of neuroendocrine prostate cancer.

24. (canceled)

25. The composition of claim 6, wherein the targeting moiety is chosen from the group consisting of the targeting peptides listed in Table 1, peptides that target the fibronectin-fibrin complex, peptides comprising the TAT sequence, the pHLIP peptide, a peptide which targets MMPs, chemokine receptor ligand CXCL12, and a peptide with the sequence WQPDTAHHWATL.

26. The composition of claim 6, wherein the chemotherapeutic agent is chosen from the group consisting of microtubule-targeting moietys (MTAs), DNA damaging agents, alkylating agents, antimetabolites, spindle poison plant alkaloids, cytotoxic/antitumor antibiotics, topoisomerase inhibitors, antibodies, photosensitizers, and kinase inhibitors.

27.-30. (canceled)

31. The composition of claim 6, wherein the targeting moiety is a SSTR-targeting moiety.
